(19) United States
(12) Patent Application Publication
(10) Pub. No.: US 2019/0322623 A1
(43) Pub. Date: Oct. 24, 2019

(54) COMPOUND, DISPLAY PANEL, AND DISPLAY APPARATUS

(71) Applicant: WuHan TianMa Micro-Electronics Co., Ltd., Wuhan (CN)

(72) Inventors: Lei Zhang, Wuhan (CN); Wei Gao, Wuhan (CN); Jinghua Niu, Wuhan (CN); Wenpeng Dai, Wuhan (CN); Dongyang Deng, Wuhan (CN); Hongyan Zhu, Wuhan (CN)

(73) Assignee: WuHan TianMa Micro-Electronics Co., Ltd., Wuhan (CN)

(21) Appl. No.: 16/503,488

(22) Filed: Jul. 4, 2019

(30) Foreign Application Priority Data
Dec. 26, 2018 (CN) 201811604397.X

(51) Int. Cl. C07D 209/86; C07D 487/04; C07D 471/04; C07D 495/04; C07D 491/048; C07D 219/08; C07D 279/18; C07D 265/38; C07D 311/82; H01L 51/00; C07F 5/02; C07F 9/53; C07F 9/572; C07F 9/655; C07F 9/6553

(52) U.S. Cl. CPC C07D 209/86; C07F 9/655354; C07D 471/04; C07D 495/04; C07D 491/048; C07D 219/08; C07D 279/18; C07D 265/38; C07D 311/82; H01L 51/0072; H01L 51/0073; H01L 51/0061; H01L 51/0067; C07F 5/027; C07F 9/5325; C07F 9/5728; C07F 9/65517; C07D 487/04

(57) ABSTRACT

Provided is a host material compound having a structure represented by Formula (I):

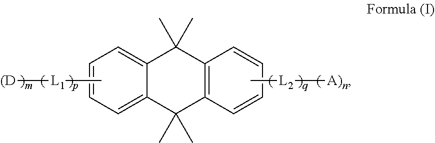

in which m and n, respectively representing the number of electron donors D and the number of electron acceptors A, are each 1, 2 or 3; p and q, respectively representing the number of the group L₁ and the number of the group L₂, are each 0, 1, or 2. D, L₁ and L₂ are each alkyl, cycloalkyl, heterocyclic group, aryl, heteroaryl, fused aryl, or fused heteroaryl; and A is selected from nitrogen-containing heterocyclic substituents, cyano-containing substituents, triaryl-boron-derived substituents, and phosphorus oxygen double bond-containing substituents. The compound has a D-(π)-σ-(π)-A structure with bipolarity, and the σ bond can interrupt an intramolecular charge transfer between D and A, so that the excited state is limited to a local excited state in moiety of D or A, and the compound has a small excited-state dipole moment.

### CROSS-REFERENCE TO RELATED APPLICATIONS

The present application claims priority to Chinese Patent Application No. 201811604397.X, filed on Dec. 26, 2018, the content of which is incorporated herein by reference in its entirety.

### FIELD

The present disclosure relates to the field of organic electroluminescent materials, and particularly, to a compound, a display panel and a display apparatus containing the compound.

### BACKGROUND

As a new generation of display technology, the organic electroluminescent devices such as organic light-emitting diodes (OLEDs) have been widely used in flat-panel displays, flexible displays, solid-state lighting and vehicle displays, due to their advantages of being ultrathin, being self-luminous, and having a wide viewing angle, fast response, high luminous efficiency, good temperature adaptability, simple manufacturing process, low driving voltage, low energy consumption and the like.

Light emitted by the OLEDs can be classified into electrofluorescence and electrophosphorescence depending upon the luminescence mechanism. Fluorescence is emission light resulted from a radiation attenuation transition of singlet excitons, and phosphorescence is emission light resulted from a radiation attenuation of triplet excitons to the ground state. According to the spin quantum statistics theory, a forming probability ratio of singlet excitons to triplet excitons is 1:3. The internal quantum efficiency of the electrofluorescent material is no more than 25%, and the external quantum efficiency thereof is generally less than 5%. Theoretically, the internal quantum efficiency of the electrophosphorescent material can reach 100%, and the external quantum efficiency thereof can be up to 20%. In 1998, Professor Yuguang Ma from Jilin University in China and Professor Forrest from Princeton University in the United States respectively reported ruthenium complexes and platinum complexes that were used as dyes doped into the light-emitting layer, successfully obtained and explained a phenomenon of phosphorescence electroluminescence for the first time, and pioneered the application of the prepared phosphorescent material to an electroluminescent device.

The long lifetime (μs) of phosphorescent heavy metal materials may lead to triplet state-triplet state quenching and concentration quenching at high current densities and further result in a degradation of device performance. Therefore, phosphorescent heavy metal materials are usually doped into suitable host materials to form a host-guest doping system. In this way, energy transfer is optimized, and luminous efficiency and lifetime are maximized. At present, the commercialization of heavy metal doping materials is mature, and it is difficult to develop alternative doping materials. Thus, developing a novel phosphorescent host material is becoming a new research topic.

### SUMMARY

In one embodiment, the present disclosure provides a compound having a D-(π)-σ-(π)-A structure. The compound has a chemical structure represented by formula (I):

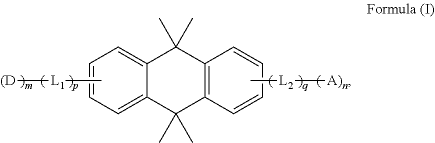

wherein D represents an electron donor, A represents an electron acceptor, m is a number of the electron donor D, n is a number of the electron acceptor A, and m and n are each 1, 2, or 3,

p is a number of the group L₁, q is a number of the group L₂, and p and q are each 0, 1, or 2,

L₁ and L₂ are each independently selected from the group consisting of a single bond, a substituted or unsubstituted C1-C20 alkylene, a substituted or unsubstituted C3-C20 cycloalkylene, a substituted or unsubstituted C3-C20 heterocycloalkylene, a substituted or unsubstituted C6-C40 arylene, a substituted or unsubstituted C4-C40 heteroarylene, a substituted or unsubstituted C10-C60 fused arylene, and a substituted or unsubstituted C10-C60 fused heteroarylene,

when p or q is 2, the two L₁ or the two L₂ are identical or different;

the electron donor D is selected from the group consisting of a substituted or unsubstituted C1-C20 alkyl, a substituted or unsubstituted C3-C20 cycloalkyl, a substituted or unsubstituted C1-C20 alkoxy, a substituted or unsubstituted C3-C20 heterocyclic group, a substituted or unsubstituted C6-C40 aryl, a substituted or unsubstituted C4-C40 heteroaryl, a substituted or unsubstituted C10-C60 fused arylene, a substituted or unsubstituted C10-C60 fused heteroarylene, a substituted or unsubstituted C12-C40 carbazolyl and a derivative group thereof, a substituted or unsubstituted C12-C40 diphenylamino and a derivative group thereof, and a substituted or unsubstituted C13-C40 acridinyl and a derivative group thereof,

when m is 2 or 3, the two or three electron donors D are identical or different,

the electron acceptor A is selected from the group consisting of nitrogen-containing heterocyclic substituents, cyano-containing substituents, triaryl-boron-derived substituents, and phosphorus oxygen double bond-containing substituents, and

when n is 2 or 3, the two or three electron acceptors A are identical or different.

In another embodiment, the present disclosure provides a display panel, including an organic light-emitting device, wherein the organic light-emitting device includes an anode, a cathode disposed oppositely to the anode, and a light-emitting layer disposed between the anode and the cathode, wherein the light-emitting layer includes a host material and a guest material, and the host material is one or more compounds of another embodiment.

In yet another embodiment, the present disclosure provides a display apparatus including the above-mentioned display panel.

The compound having the D-(π)-σ-(π)-A structure according to the present disclosure is a bipolar material, which can replace the conventional D-π-A skeleton known in the prior art. The conventional D-π-A bipolar material has a strong intramolecular charge transfer, which may result in a large dipole moment μs. The D-(π)-σ-(π)-A structure of the compound according to the present disclosure has bipolarity, and the intermediate 6 bond can effectively interrupt the intramolecular charge transfer between the electron donor D and the electron acceptor A, so that the excited state is limited to a local excited state in moiety of the electron donor D or the electron acceptor A, and thus the compound has a small excited-state dipole moment. In this way, the compound, when used as host material of a light-emitting layer of an OLED device, can effectively reduce an efficiency roll-off of a blue light material and enhance the brightness and luminous efficiency.

The compound according to the present disclosure, which is used as the host material in an electroluminescent device, has a high triplet energy level E_{T}, a large molecular density, a high glass transition temperature and a high molecular thermal stability, and thus can effectively improve an equilibrium migration of carriers and widen a recombination area of excitons. In this regard, the external quantum efficiency (EQE) and service life of the device are effectively enhanced. Therefore, the compound according to the present disclosure can be well applied in the electroluminescent device field.

### BRIEF DESCRIPTION OF THE DRAWINGS

FIG. 1 is a chemical formula of a compound according to the present disclosure;

FIG. 2 is a structural schematic diagram of an OLED device according to an embodiment of the present disclosure; and

FIG. 3 is a schematic diagram of a display apparatus according to an embodiment of the present disclosure.

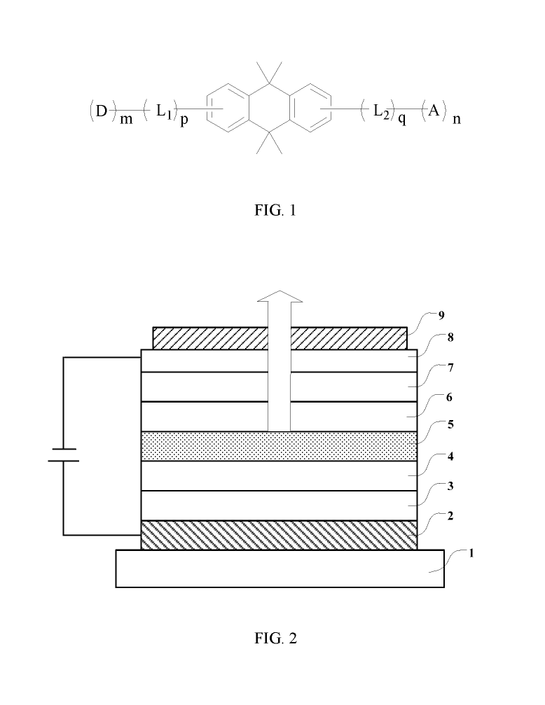

### DESCRIPTION OF EMBODIMENTS

The present disclosure is described in detail with the aid of embodiments and comparative examples. The following embodiments are merely used to illustrate the present disclosure, but not intended to limit the scope of the present disclosure. Any modification or equivalent replacement with respect to the embodiments of the present disclosure, without departing from the scope of the present disclosure, shall fall into the protection scope of the present disclosure.

In one embodiment, the present disclosure provides a compound having a chemical structure represented by Formula (I):

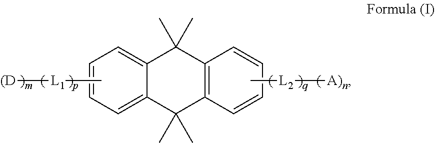

in which D represents an electron donor, A represents an electron acceptor, m is a number of the electron donor D, n is a number of the electron acceptor A, and m and n are each independently 1, 2, or 3, p is a number of the group L₁, q is a number of the group L₂, and p and q are each independently 0, 1, or 2,

L₁ and L₂ are each independently selected from the group consisting of a single bond, a substituted or unsubstituted C1-C20 alkylene, a substituted or unsubstituted C3-C20 cycloalkylene, a substituted or unsubstituted C3-C20 heterocycloalkylene, a substituted or unsubstituted C6-C40 arylene, a substituted or unsubstituted C4-C40 heteroarylene, a substituted or unsubstituted C10-C60 fused arylene, and a substituted or unsubstituted C10-C60 fused heteroarylene,

the electron donor D is selected from the group consisting of a substituted or unsubstituted C1-C20 alkyl, a substituted or unsubstituted C3-C20 cycloalkyl, a substituted or unsubstituted C1-C20 alkoxy, a substituted or unsubstituted C3-C20 heterocyclic group, a substituted or unsubstituted C6-C40 aryl, a substituted or unsubstituted C4-C40 heteroaryl, a substituted or unsubstituted C10-C60 fused arylene, a substituted or unsubstituted C10-C60 fused heteroarylene, a substituted or unsubstituted C12-C40 carbazolyl and a derivative group thereof, a substituted or unsubstituted C12-C40 diphenylamino and a derivative group thereof, and a substituted or unsubstituted C13-C40 acridinyl and a derivative group thereof, and

the electron acceptor A is selected from the group consisting of nitrogen-containing heterocyclic substituents, cyano-containing substituents, triaryl-boron-derived substituents, and phosphorus oxygen double bond-containing substituents.

According to an embodiment of the compound of the present disclosure, the electron donor D is selected from the following groups:

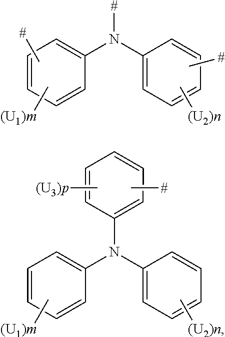

in which m, n and p are each independently 0, 1, 2, or 3,

U₁, U₂ and U₃ are each independently selected from the group consisting of hydrogen, a substituted or unsubstituted C1-C30 alkyl, a substituted or unsubstituted silicylene, a substituted or unsubstituted C3-C20 cycloalkyl, a substituted or unsubstituted C1-C30 alkoxy, a substituted or unsubstituted C6-C30 aryl, and a substituted or unsubstituted C10-C30 fused aryl, and

# represents a bonding position.

According to an embodiment of the compound of the present disclosure, the electron donor D is selected from the following groups:

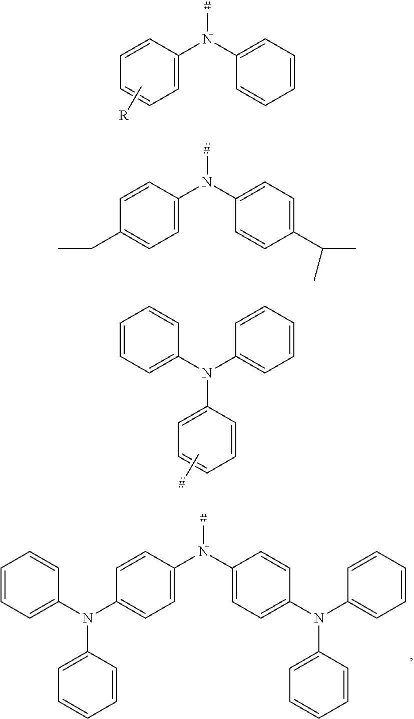

in which R is selected from the group consisting of hydrogen, a substituted or unsubstituted C1-C20 alkyl, a substituted or unsubstituted silicylene, a substituted or unsubstituted C3-C20 cycloalkyl, a substituted or unsubstituted C1-C20 alkoxy, a substituted or unsubstituted C3-C20 heterocyclic group, a substituted or unsubstituted C6-C40 aryl, a substituted or unsubstituted C10-C30 fused aryl, and a substituted or unsubstituted C4-C40 heteroaryl.

According to an embodiment of the compound of the present disclosure, the electron donor D is selected from the following groups:

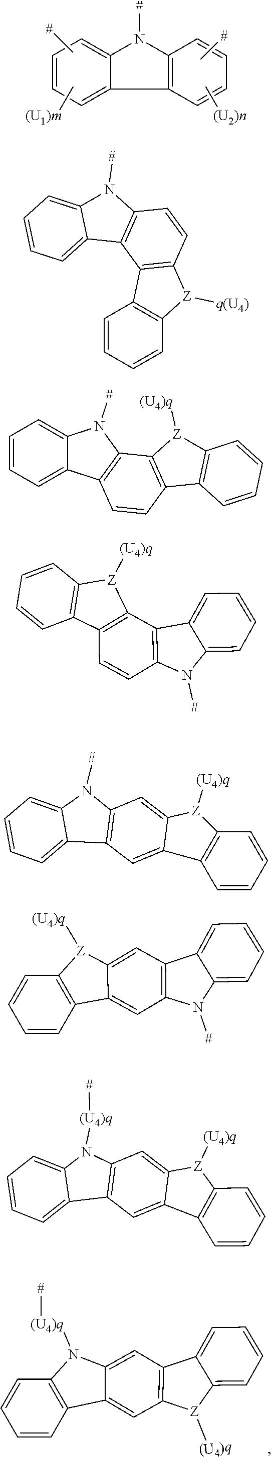

in which Z is carbon, nitrogen, oxygen, sulfur, or silicon,

q is 0, 1, 2, or 3,

U₁, U₂ and U₄ are each independently selected from the group consisting of hydrogen, a substituted or unsubstituted C1-C30 alkyl, a substituted or unsubstituted silicylene, a substituted or unsubstituted C3-C20 cycloalkyl, a substituted or unsubstituted C1-C30 alkoxy, a substituted or unsubstituted C6-C30 aryl, and a substituted or unsubstituted C10-C30 fused aryl,

when Z is oxygen or sulfur, q is 0, and

# represents a bonding position.

According to an embodiment of the compound of the present disclosure, the electron donor D is selected from the following groups:

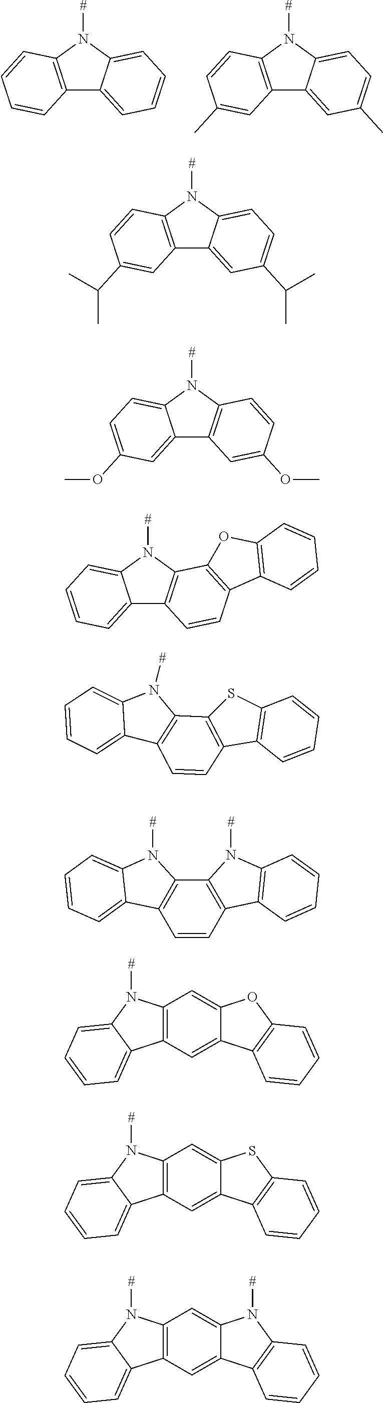

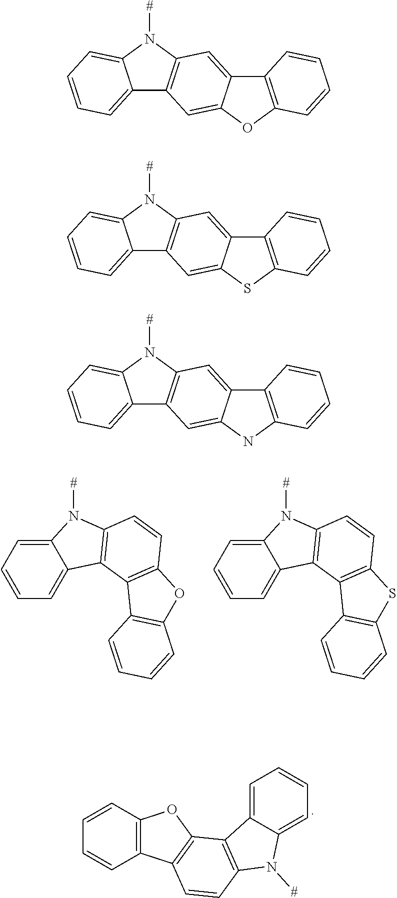

According to an embodiment of the compound of the present disclosure, the electron donor D is selected from the following groups:

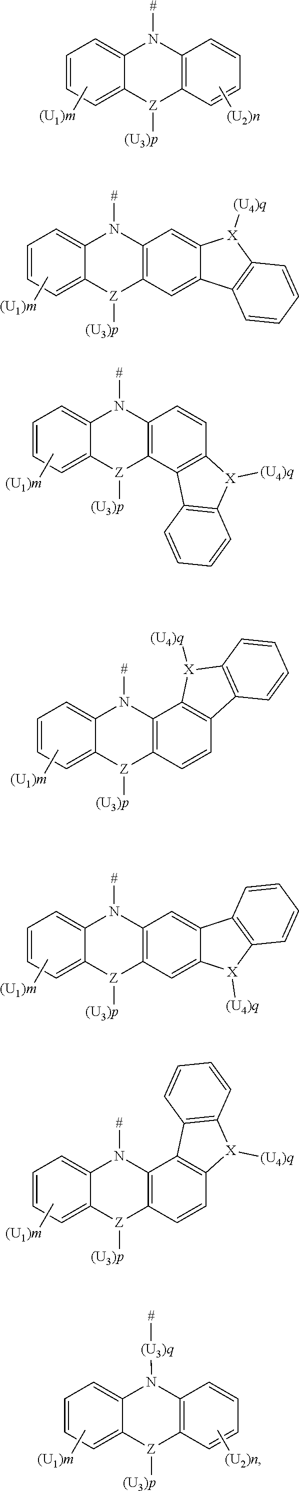

in which Z is carbon, nitrogen, oxygen, sulfur, or silicon,

X is carbon, nitrogen, oxygen, or sulfur,

m, n, p and p are each independently 0, 1, 2, or 3,

U₁, U₂, U₃ and U₄ are each independently selected from the group consisting of hydrogen, a substituted or unsubstituted C1-C30 alkyl, a substituted or unsubstituted silicylene, a substituted or unsubstituted C3-C20 cycloalkyl, a substituted or unsubstituted C1-C30 alkoxy, a substituted or unsubstituted C6-C30 aryl, and a substituted or unsubstituted C10-C30 fused aryl,

when Z is oxygen or sulfur, p is 0,

when X is oxygen or sulfur, q is 0, and

# represents a bonding position.

According to an embodiment of the compound of the present disclosure, the electron donor D is selected from the following groups:

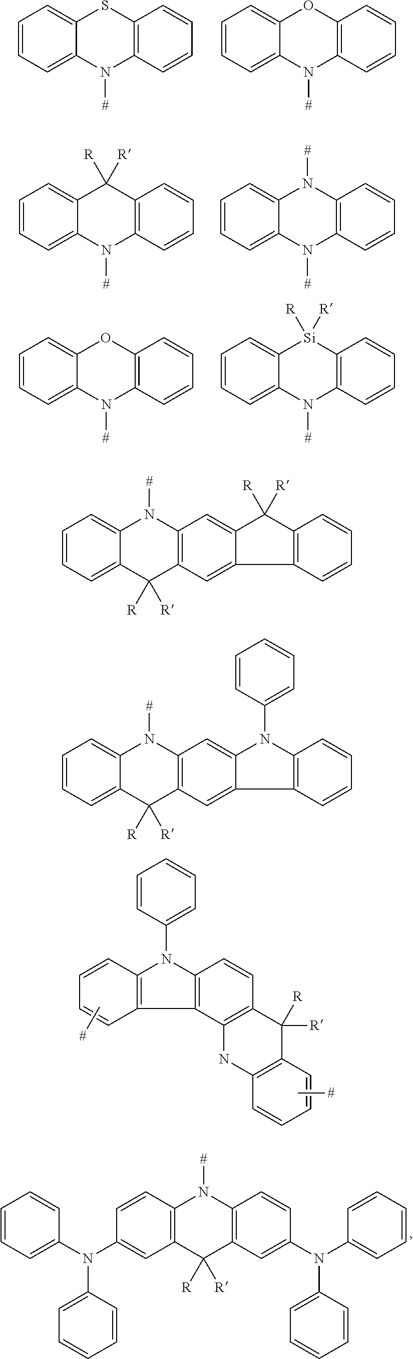

in which R and R′ are each independently selected from the group consisting of hydrogen, a substituted or unsubstituted C1-C20 alkyl, a substituted or unsubstituted C3-C20 cycloalkyl, a substituted or unsubstituted C1-C20 alkoxy, a substituted or unsubstituted C3-C20 heterocyclic group, a substituted or unsubstituted C6-C40 aryl, and a substituted or unsubstituted C4-C40 heteroaryl.

According to an embodiment of the compound of the present disclosure, the electron acceptor A is selected from the following groups:

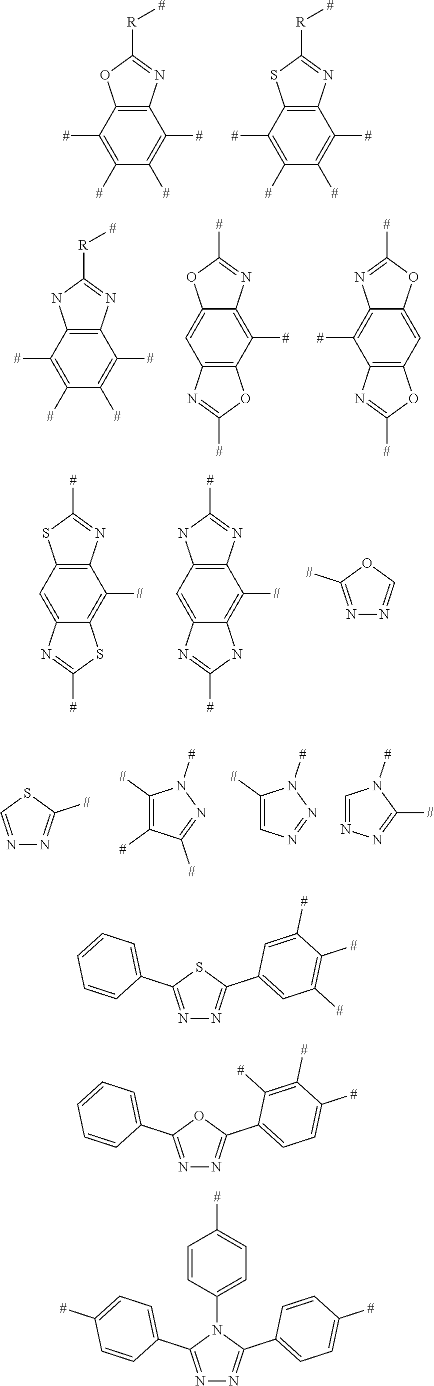

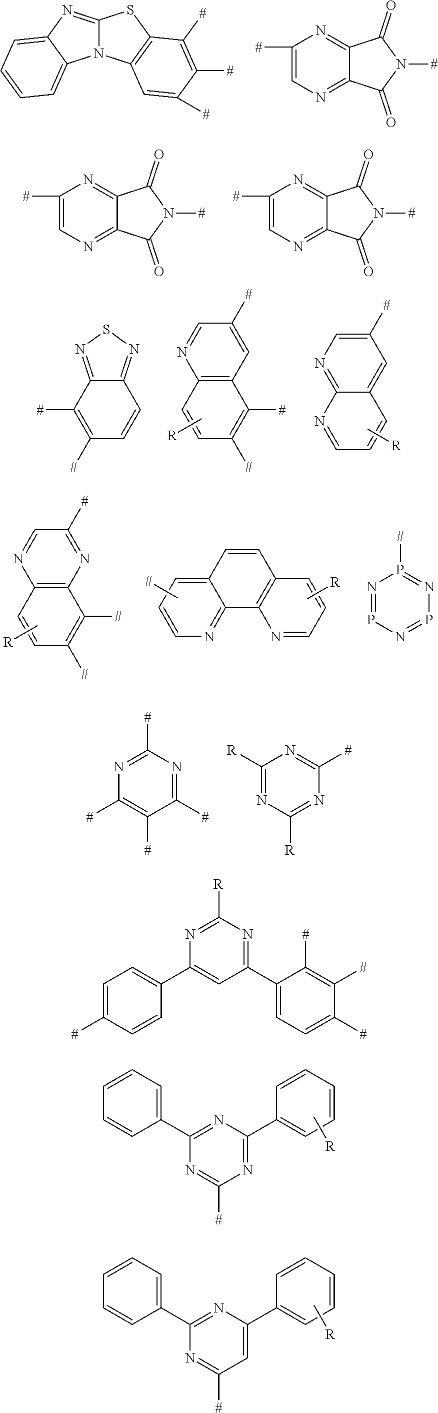

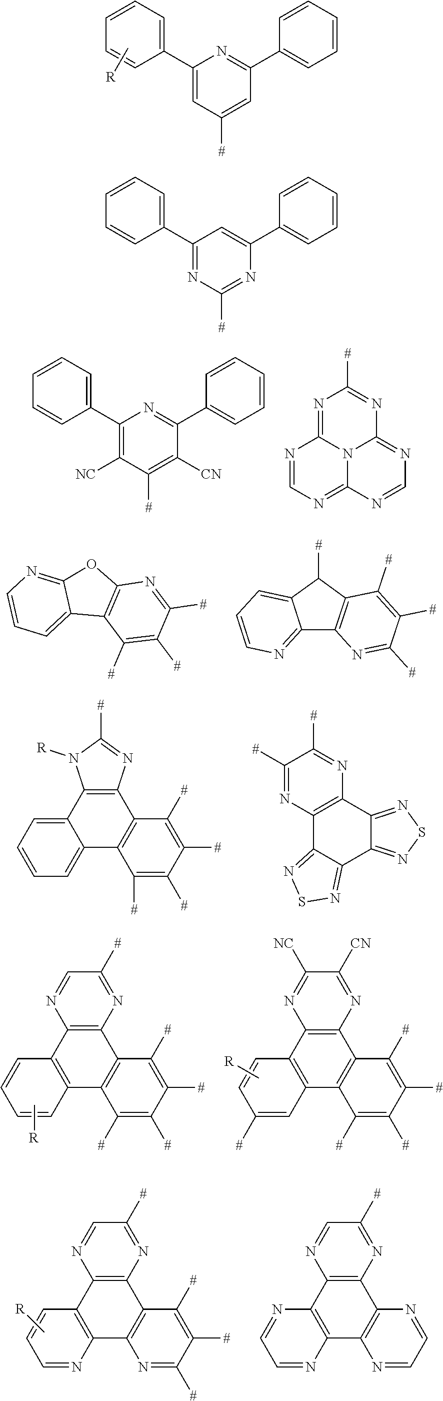

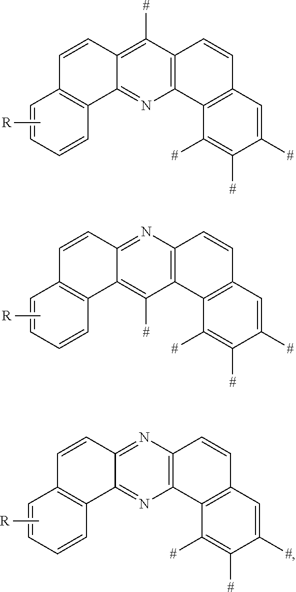

in which R is hydrogen, a C1-C20 alkyl, a C1-C20 alkoxy, a C4-C8 cycloalkyl, a C6-C40 aryl, or a C4-C40 heteroaryl, and

# represents a bonding position.

According to an embodiment of the compound of the present disclosure, the electron acceptor A is selected from the following groups:

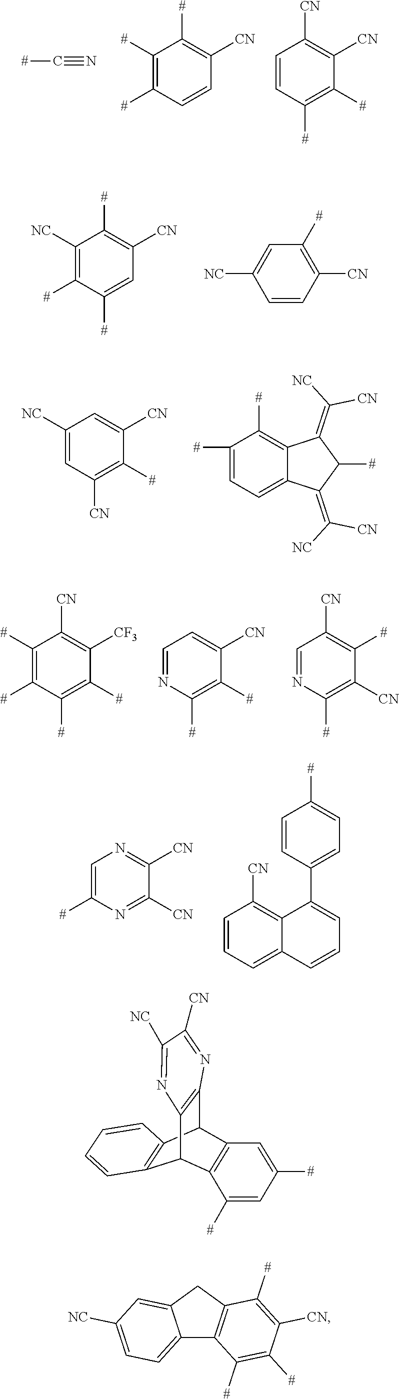

in which # represents a bonding position.

According to an embodiment of the compound of the present disclosure, the electron acceptor A is selected from the following groups:

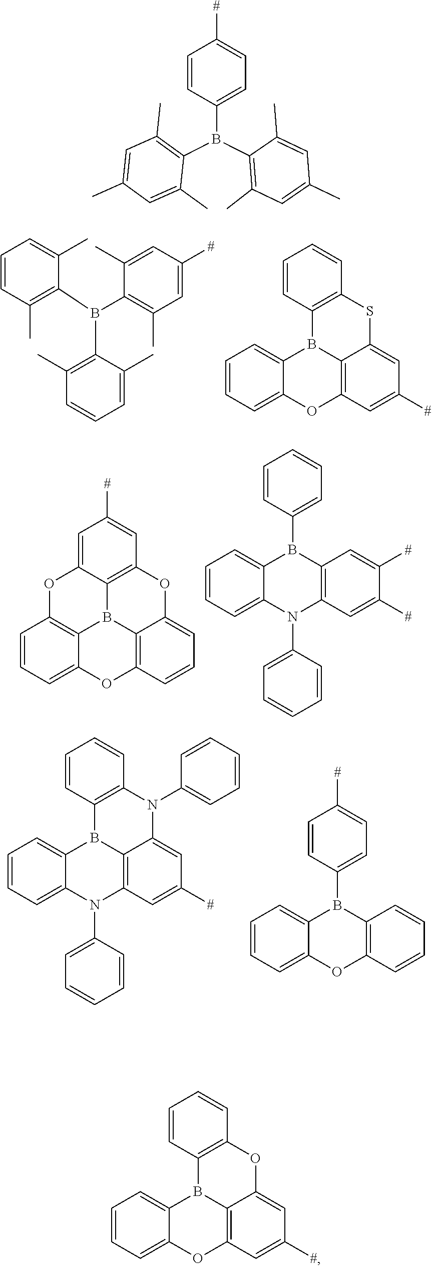

in which # represents a bonding position.

According to an embodiment of the compound of the present disclosure, the electron acceptor A is selected from the following groups:

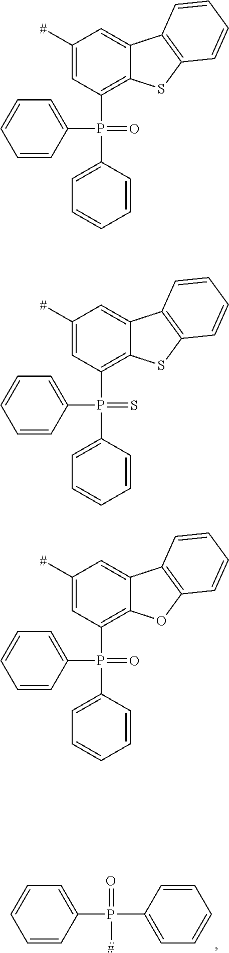

in which # represents a bonding position.

According to an embodiment of the compound of the present disclosure, the compound is selected from the following compounds:

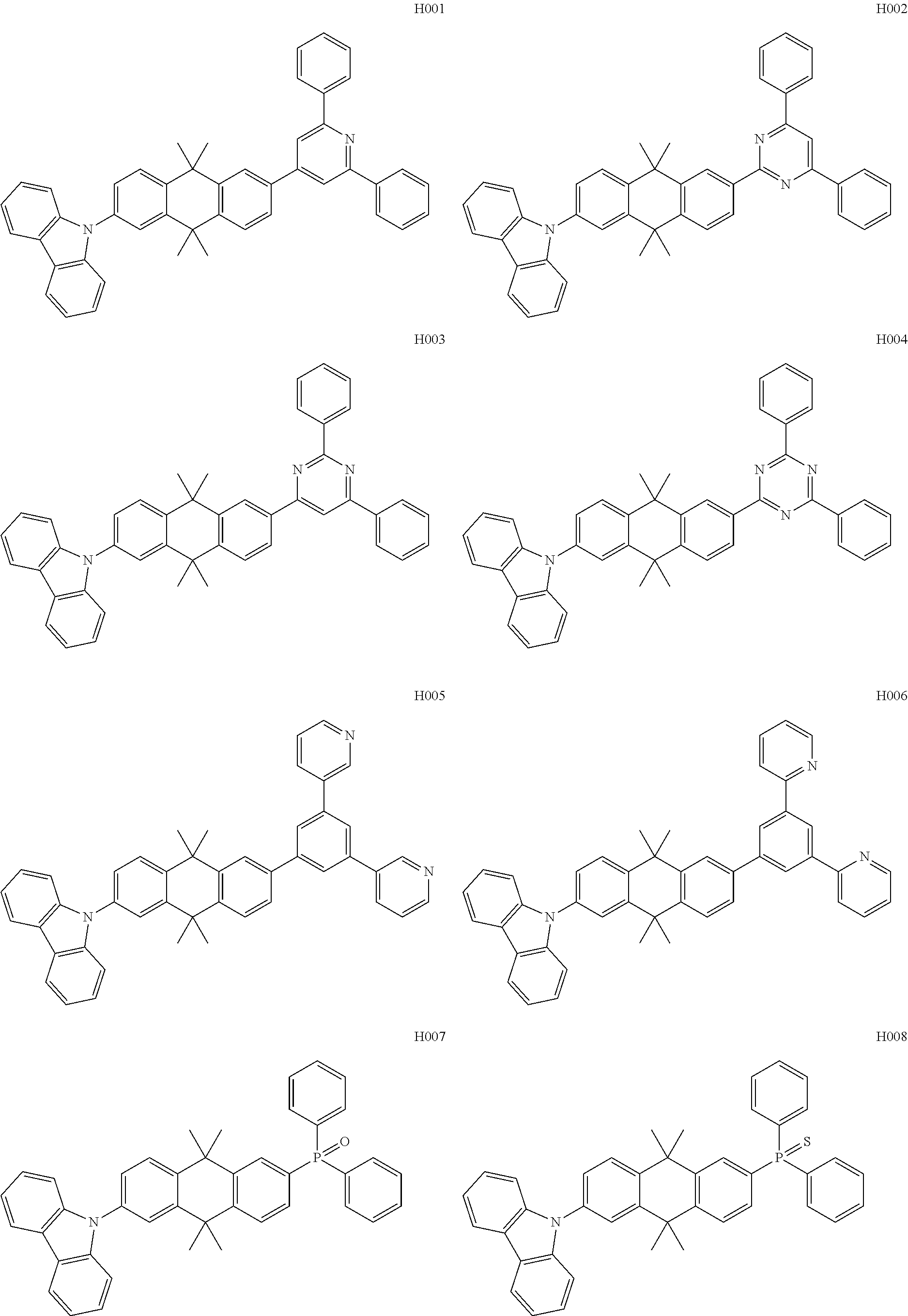

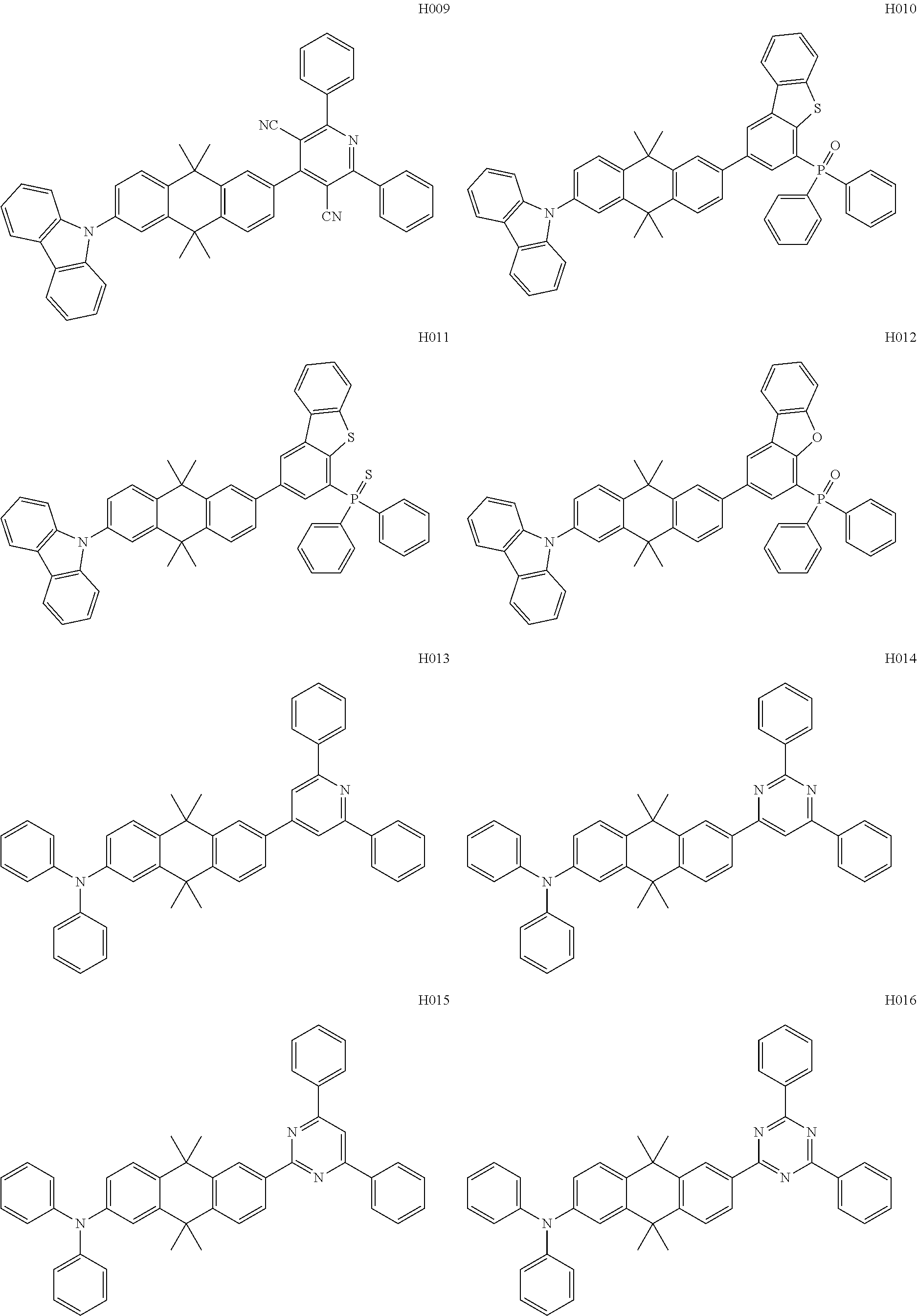

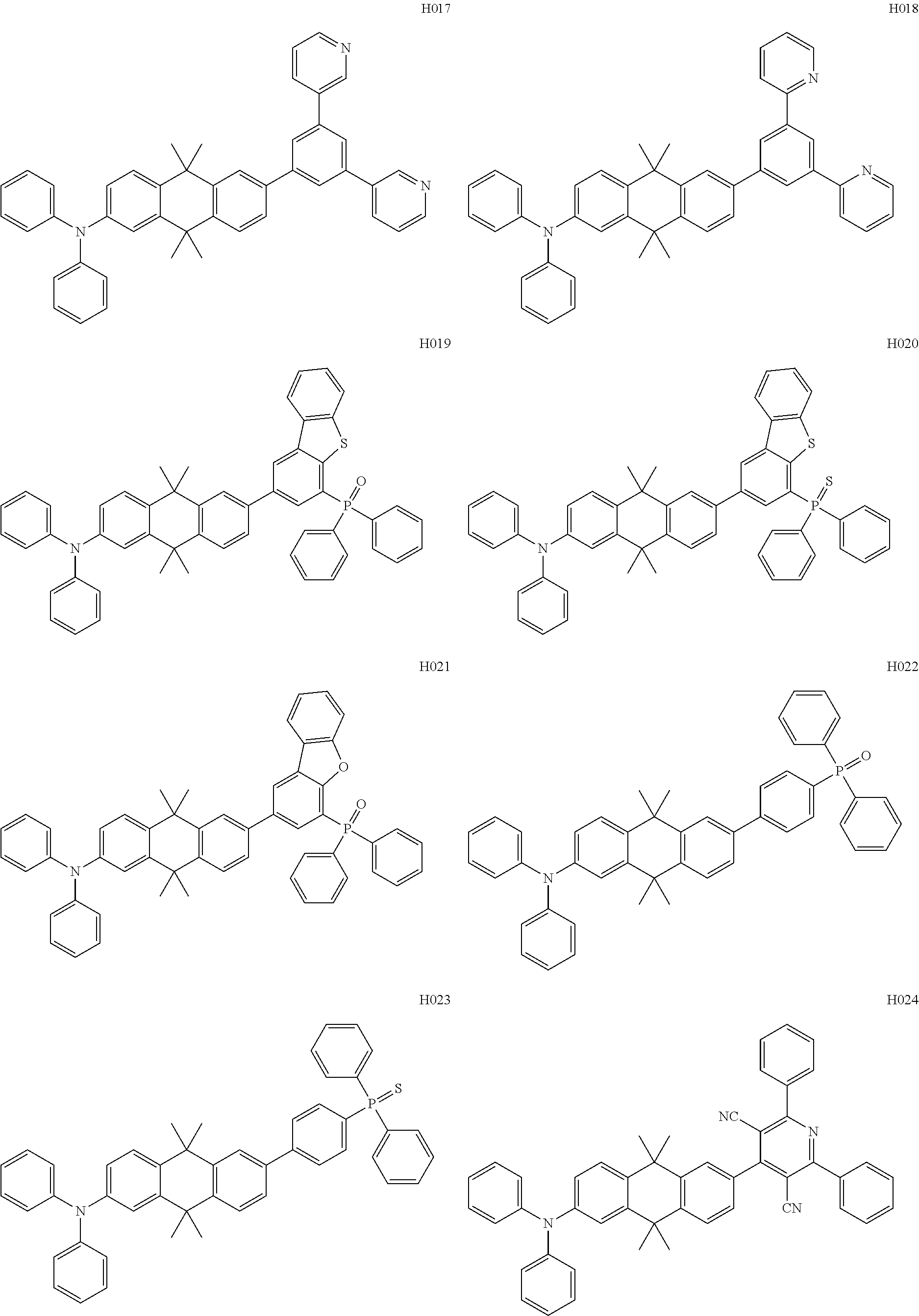

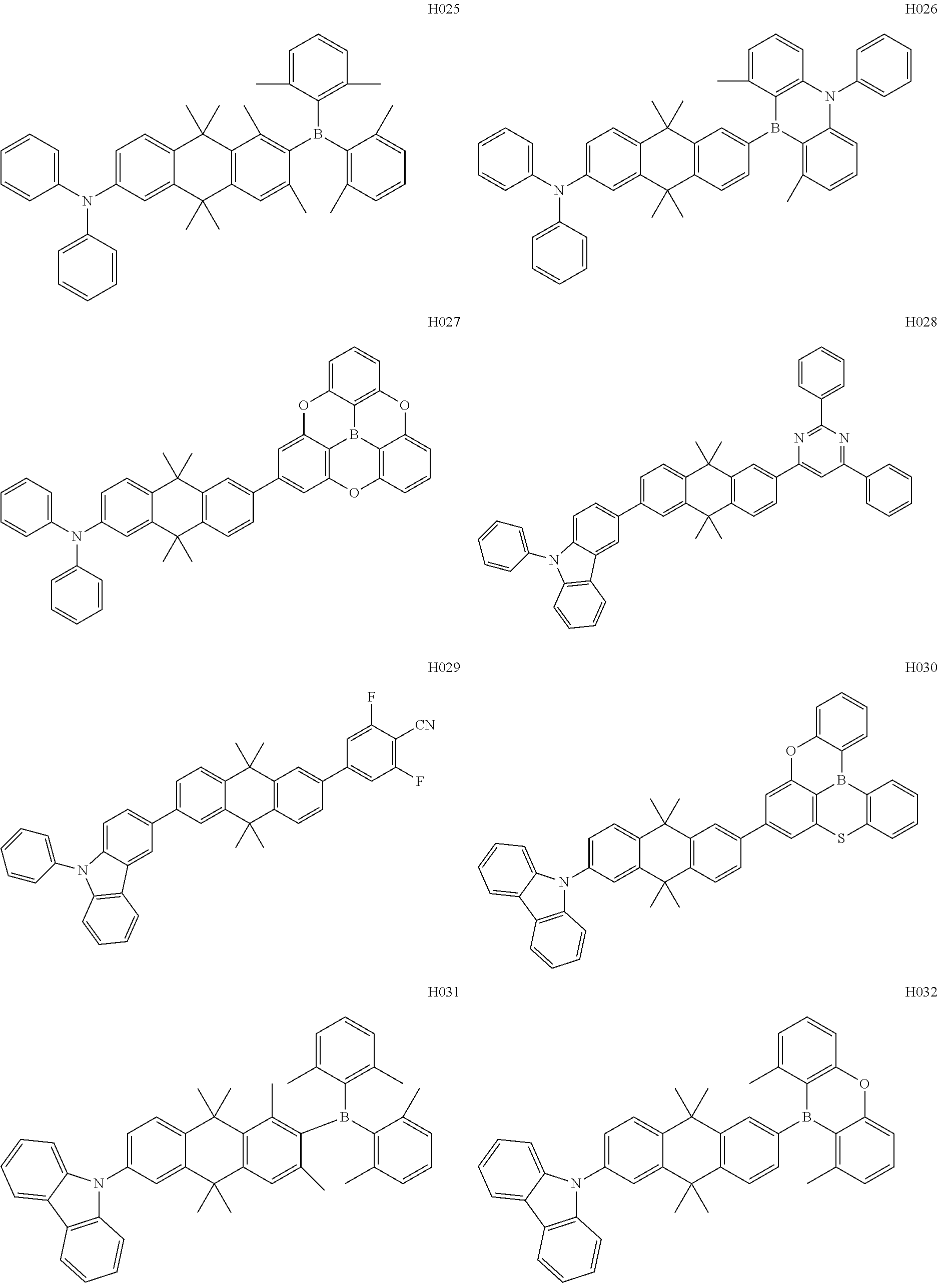

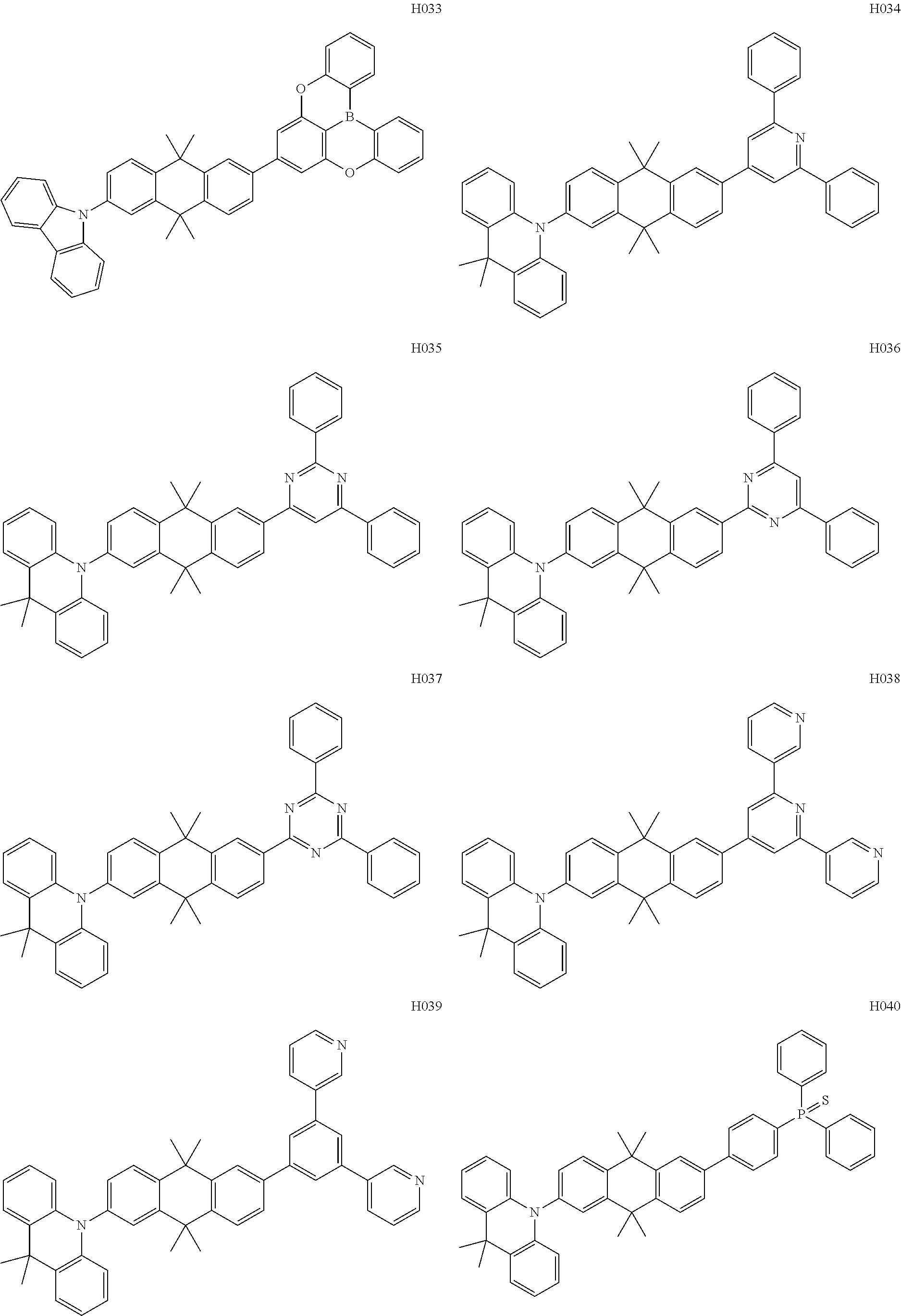

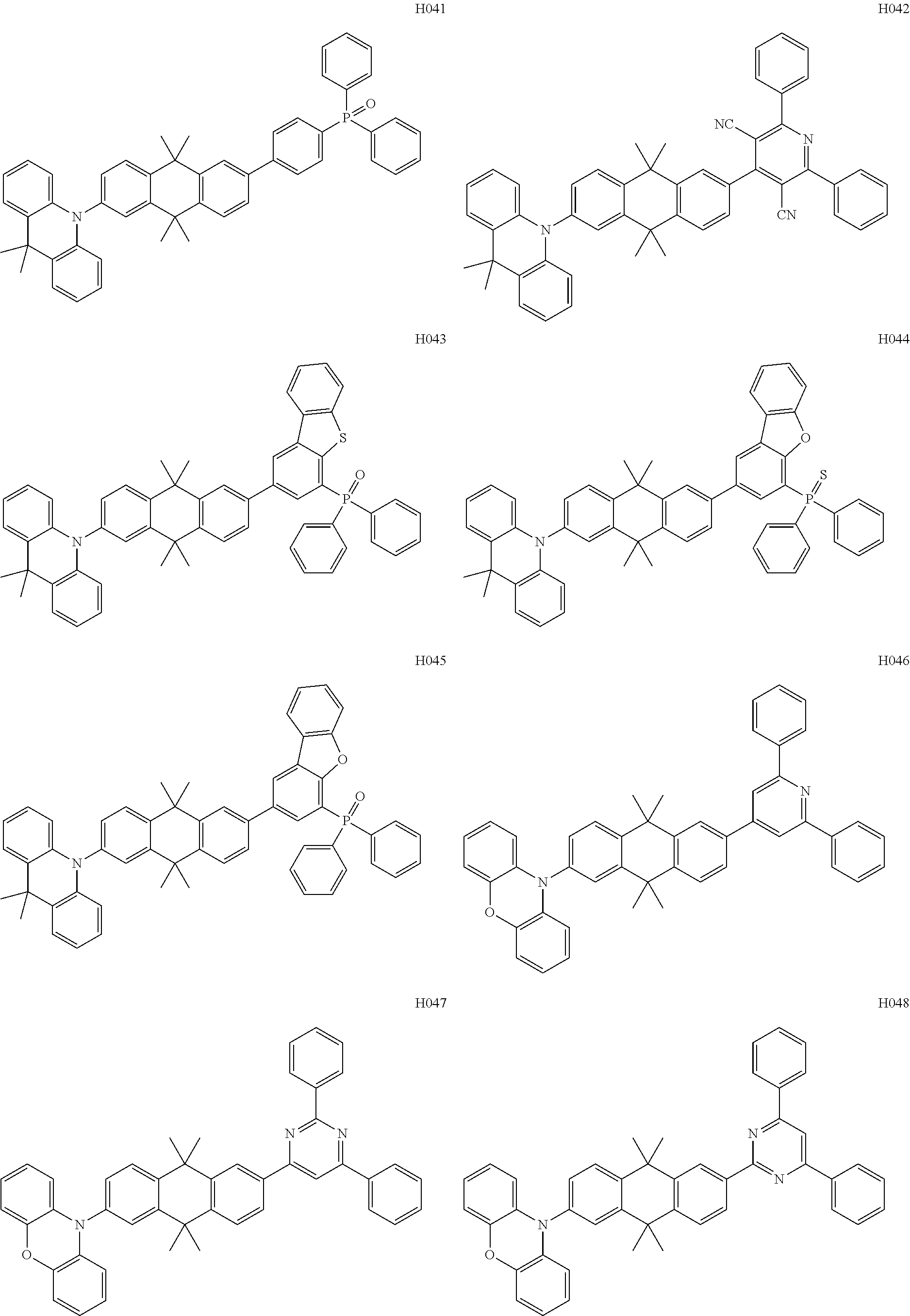

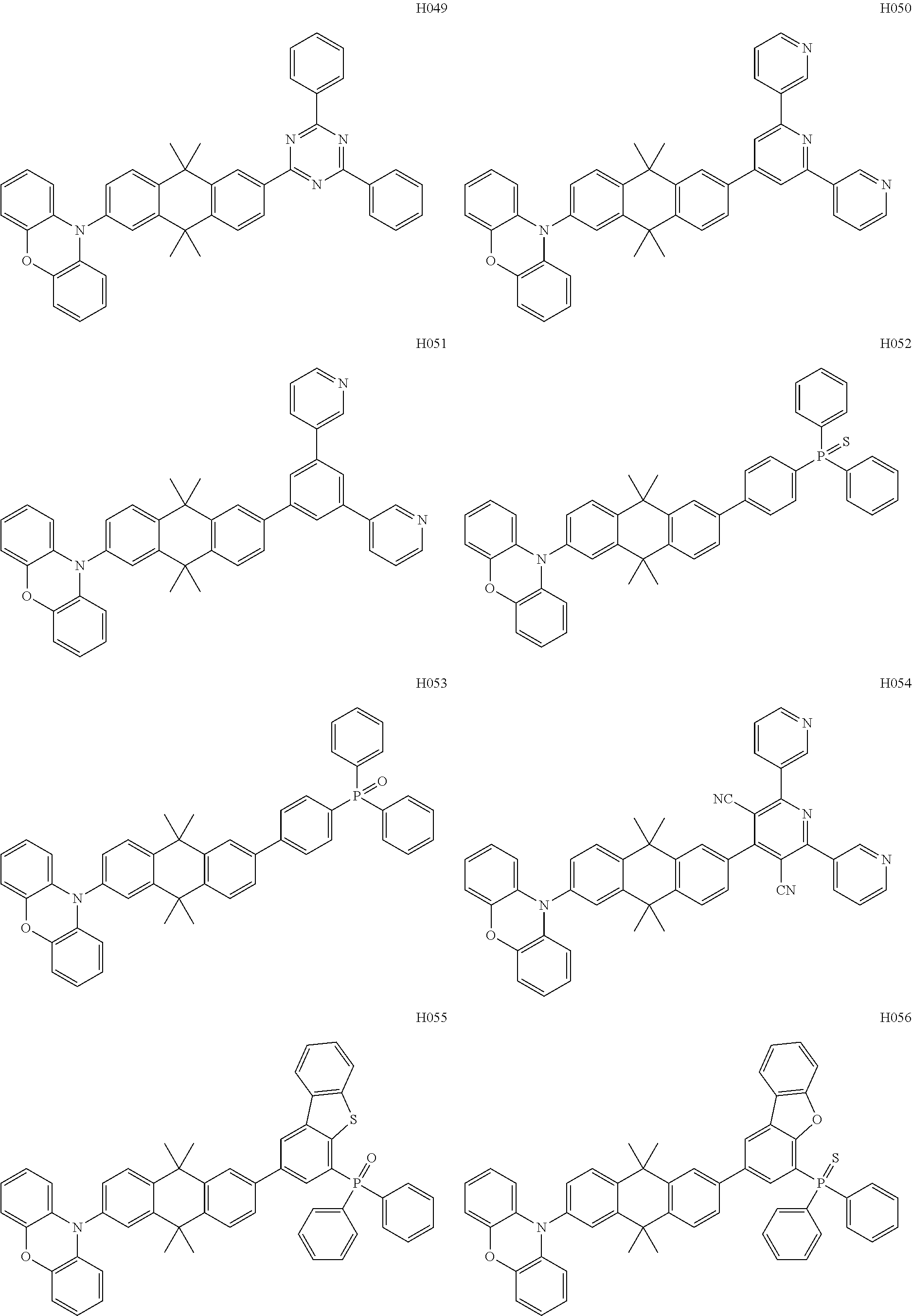

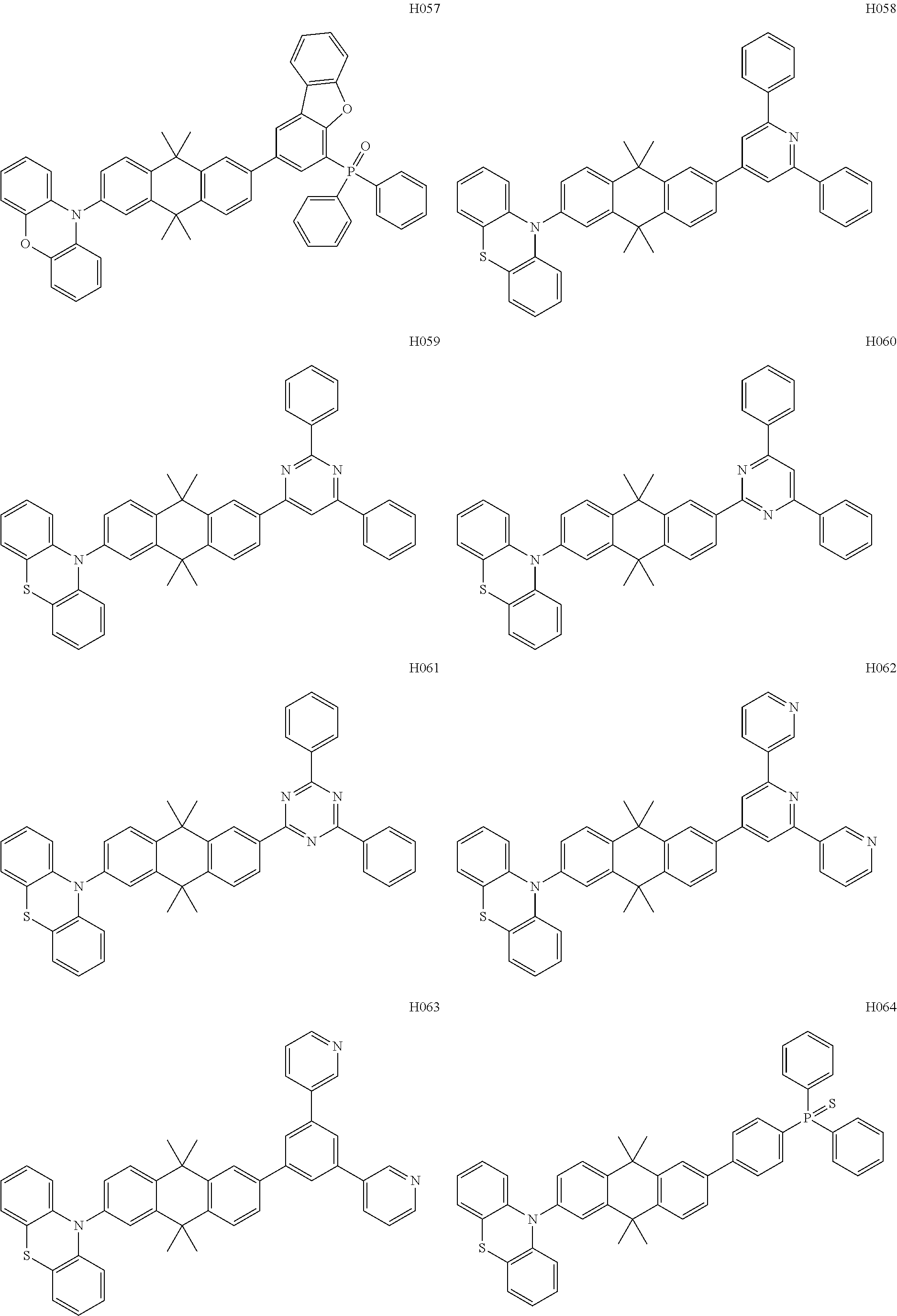

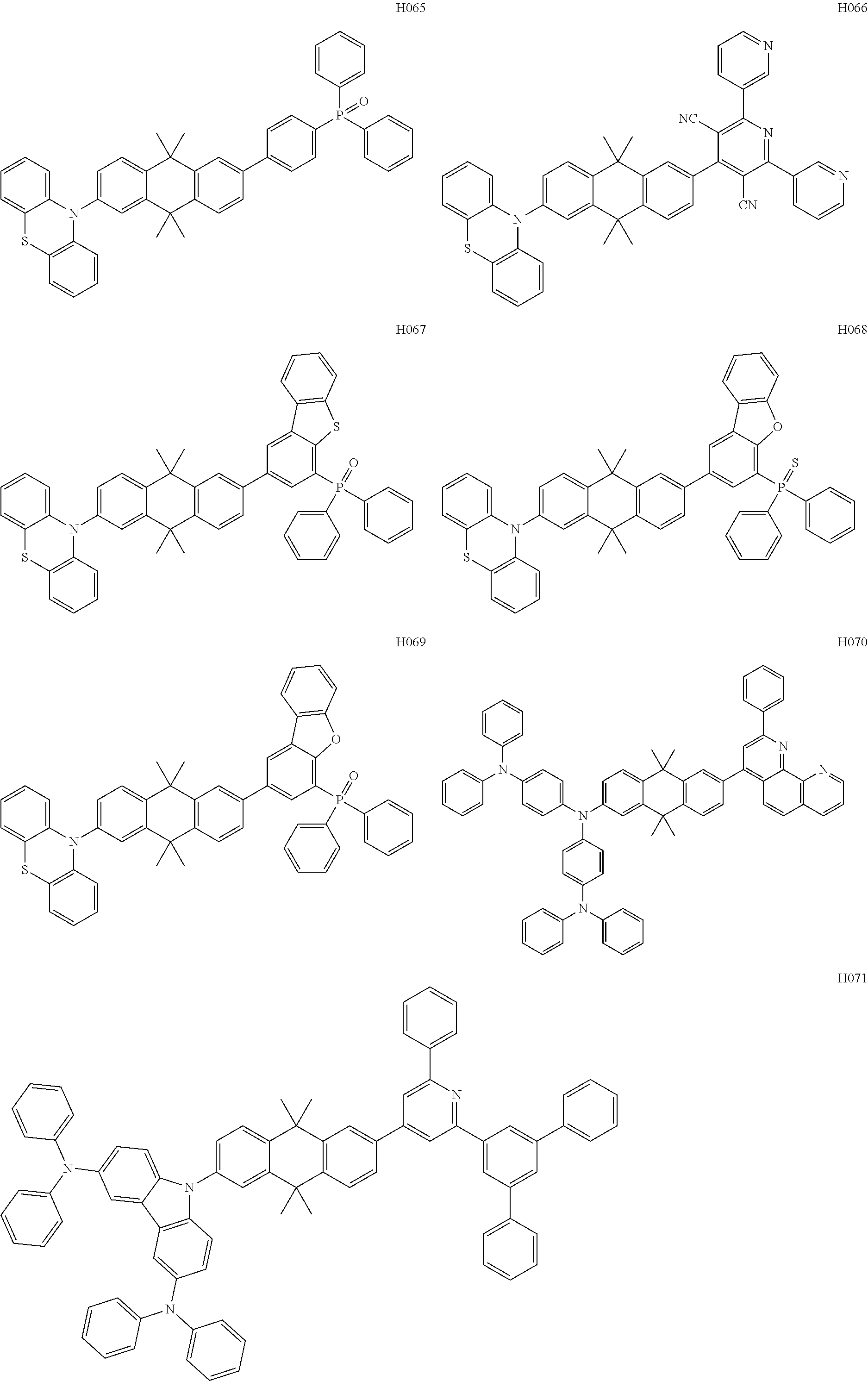

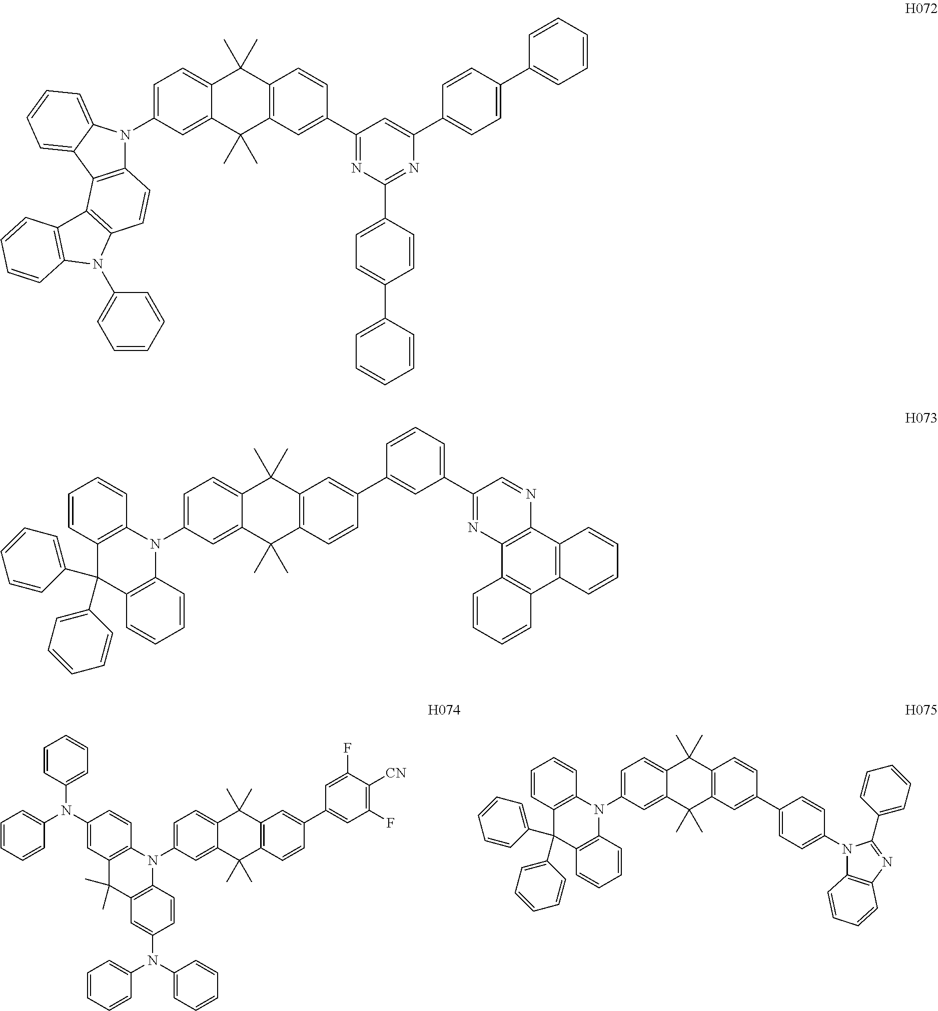 [SMILES: CC1(C)c2ccc(-c3cc(-c4ccc(-c5ccccc5)cc4)nc(-c4ccc(-c5ccccc5)cc4)n3)cc2C(C)(C)c2cc(-n3c4ccccc4c4c5c6ccccc6n(-c6ccccc6)c5ccc43)ccc21 ; CC1(C)c2ccc(N3c4ccccc4C(c4ccccc4)(c4ccccc4)c4ccccc43)cc2C(C)(C)c2ccc(-c3cccc(-c4cnc5c6ccccc6c6ccccc6c5n4)c3)cc21 ; CC1(C)c2cc(N(c3ccccc3)c3ccccc3)ccc2N(c2ccc3c(c2)C(C)(C)c2ccc(-c4cc(F)c(C#N)c(F)c4)cc2C3(C)C)c2ccc(N(c3ccccc3)c3ccccc3)cc21 ; CC1(C)c2ccc(-c3ccc(-n4c(-c5ccccc5)nc5ccccc54)cc3)cc2C(C)(C)c2cc(N3c4ccccc4C(c4ccccc4)(c4ccccc4)c4ccccc43)ccc21]

The present disclosure further provides a display panel including an organic light-emitting device. The organic light-emitting device includes an anode, a cathode disposed oppositely to the anode, a light-emitting layer disposed between the anode and the cathode. The light-emitting layer includes a host material and a guest material. The host material is one or more compounds according to the present disclosure.

In the display panel according to the present disclosure, a singlet energy level S1 of the host material is higher than a singlet energy level S1 of the guest material, and an energy difference between the singlet energy level S1 of the host material and the singlet energy level S1 of the guest material is less than 0.8 eV. In addition, a triplet energy level T1 of the host material is higher than a triplet energy level T1 of the guest material, and an energy difference between the triplet energy level T1 of the host material and the triplet energy level T1 of the guest material is less than 0.4 eV.

In the display panel according to the present disclosure, when the host material of the light-emitting layer is a red-light-emitting material, a triplet energy level T1 of the red-light-emitting material has a lowest value as 2.2 eV.

In the display panel according to the present disclosure, when the host material of the light-emitting layer is a green-light-emitting material, a triplet energy level T1 of the green-light-emitting material has a lowest value as 2.5 eV.

In the display panel according to the present disclosure, when the host material of the light-emitting layer is a blue-light-emitting material, a triplet energy level T1 of the blue-light-emitting material has a lowest value as 2.7 eV.

According to an embodiment of the display panel of the present disclosure, the organic light-emitting device further includes one or more of a hole injection layer, a hole transmission layer, an electron blocking layer, a hole blocking layer, an electron transmission layer, and an electron injection layer.

According to an embodiment of the display panel of the present disclosure, the display panel includes an organic light-emitting device. The organic light-emitting device includes an anode, a cathode disposed oppositely to the anode, a capping layer disposed on a side of the cathode facing away from the anode, and an organic layer disposed between the anode and the cathode. The organic layer includes an electron transmission layer, a hole transmission layer, and a light-emitting layer. At least one of the capping layer, the electron transmission layer, the hole transmission layer, and the light-emitting layer is made of the compound according to the present disclosure.

In the display panel provided by the present disclosure, the anode of the organic light-emitting device can be made of a material selected from a group consisting of metals, such as copper, gold, silver, iron, chromium, nickel, manganese, palladium, platinum, etc., and alloys thereof; metal oxides, such as indium oxide, zinc oxide, indium tin oxide (ITO), indium zinc oxide (IZO), and the like; and conductive polymers, such as polyaniline, polypyrrole, poly(3-methylthiophene) and the like. In addition to the above-mentioned anode materials and the combinations thereof that are conductive to injecting holes, the anode also can be made of other suitable material known in the related art.

In the display panel provided by the present disclosure, the cathode of the organic light-emitting device can be made of a material selected from metals, such as aluminum, magnesium, silver, indium, tin, titanium, etc., and alloys thereof; and multi-layered metal materials, such as LiF/Al, LiO₂/Al, BaF₂/Al, and the like. In addition to the above-mentioned cathode materials and the combinations thereof that are conductive to injecting electrons, the cathode also can be made of other suitable material known in the related art.

According to an embodiment of the present disclosure, the organic light-emitting device of the display panel can be manufactured by forming an anode on a transparent or opaque smooth substrate, forming a thin organic layer on the anode, and further forming a cathode on the thin organic layer. The thin organic layer can be formed by a known film forming method such as vapor deposition, sputtering, spin coating, dipping, ion plating, and the like. Finally, an organic optical capping layer CPL (covering layer) was formed on the cathode. The optical capping layer CPL can be made of the compound according to the present disclosure. The optical capping layer CPL can be prepared by vapor deposition or solution processing method. The solution processing method include ink jet printing, spin coating, knife coating, screen printing, roll-to-roll printing, and the like.

The synthesis of several exemplary compounds is described below.

### Example 1

### Synthesis of Compound H003

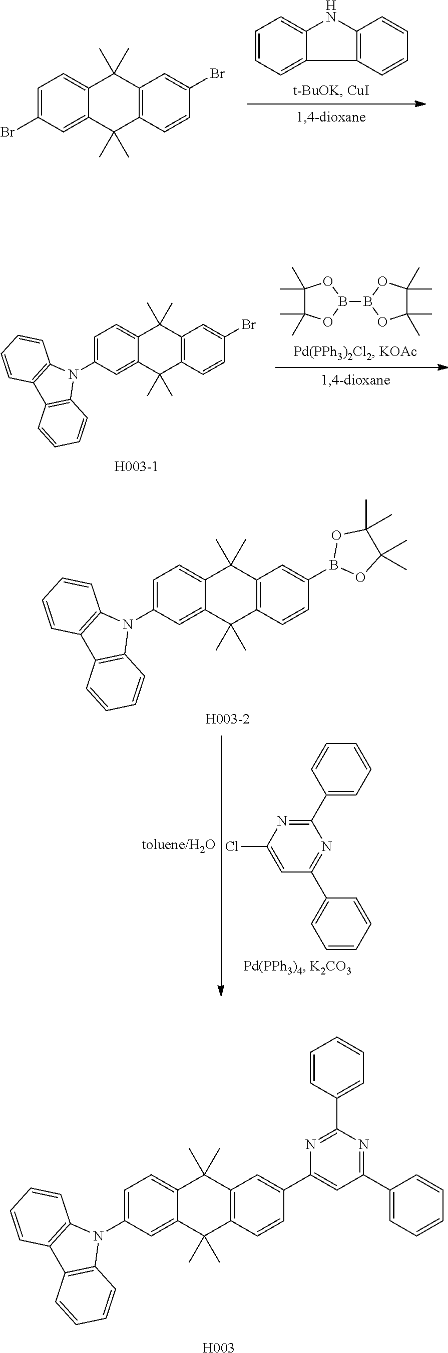

2,6-dibromo-9,9,10,10-tetramethyl-9,10-dihydroanthracene (15 mmol), copper iodide (15 mmol), potassium tert-butoxide (65 mmol), 1,2-diamino cyclohexane (12 mmol) and 9H-carbazole (25 mmol) were added to dry 1,4-dioxane (400 mL) in a round bottom flask (250 mL), and the mixture was refluxed under nitrogen atmosphere for 48 hours. The obtained intermediate was cooled to room temperature, added to water, and then filtered through a diatomite pad. The filtrate was extracted with dichloromethane, then washed with water and dried over anhydrous magnesium sulfate. A crude product was obtained after filtration and evaporation, and then purified by silica gel column chromatography to yield an intermediate product H003-1.

The intermediate product H003-1 (15 mmol) and potassium acetate (40 mmol) were mixed with dry 1,4-dioxane (60 mL), Pd(PPh₃)₂Cl₂ (0.4 mmol) and bis(pinacolato)diboron (25 mmol) in a round bottom flask (250 mL). The mixture was stirred at 90° C. for 48 hours under nitrogen atmosphere. The obtained intermediate was cooled to room temperature, added to water, and then filtered through a diatomite pad. The filtrate was extracted with dichloromethane, then washed with water and dried over anhydrous magnesium sulfate. A crude product was obtained after filtration and evaporation, and then purified by silica gel column chromatography to yield an intermediate product H003-2.

The intermediate product H003-2 (10 mmol), 4-chloro-2,6-diphenylpyrimidine (12 mmol) and Pd(PPh₃)₄ (0.3 mmol) were added to a mixture of toluene (30 mL)/ethanol (20 mL) and an aqueous solution (10 mL) of potassium carbonate (12 mmol) in a round bottom flask (250 mL). The obtained mixture was refluxed for 12 hours under nitrogen atmosphere, added to water after being cooled to room temperature, and then filtered through a diatomite pad. The filtrate was extracted with dichloromethane, then washed with water and dried over anhydrous magnesium sulfate. A crude product was obtained after filtration and evaporation, and then purified by silica gel column chromatography to yield a final product H003.

Elemental analysis of the Compound H003 (molecular formula C₄₆H₃₇N₃): theoretical values: C, 87.45; H, 5.90; N, 6.65; tested values: C, 87.45; H, 5.91; N, 6.64. Liquid chromatography-mass spectrometry ESI-MS (m/z) (M+): theoretical value: 631.30; tested value: 631.81.

### Example 2

### Synthesis of Compound H017

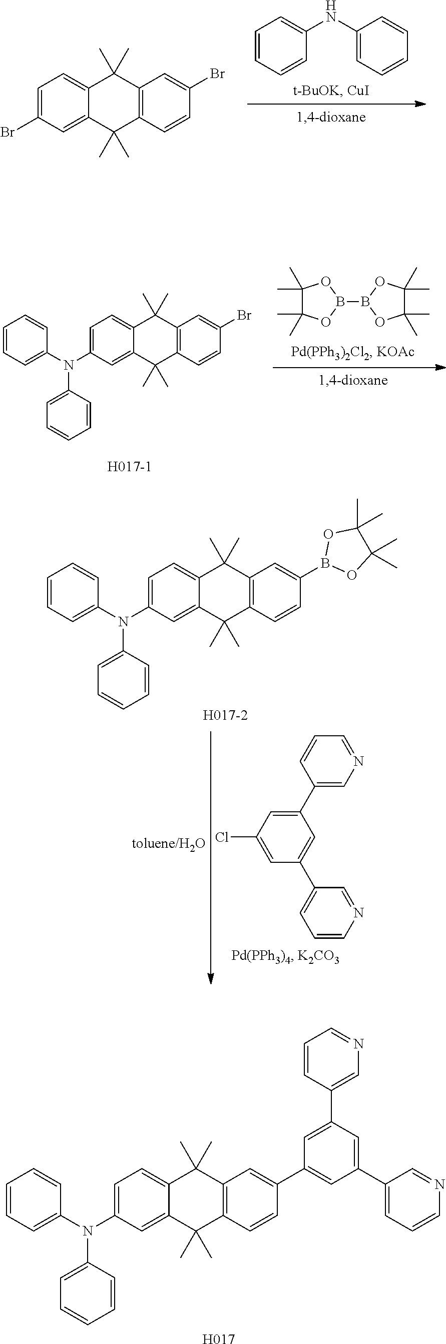

2,6-dibromo-9,9,10,10-tetramethyl-9,10-dihydroanthracene (15 mmol), copper iodide (15 mmol), potassium tert-butoxide (65 mmol), 1,2-diamino cyclohexane (12 mmol) and diarylamine (25 mmol) were added to dry 1,4-dioxane (400 mL) in a round bottom flask (250 mL), and the mixture was refluxed under nitrogen atmosphere for 48 hours. The obtained intermediate was cooled to room temperature, added to water, and then filtered through a diatomite pad. The filtrate was extracted with dichloromethane, then washed with water and dried over anhydrous magnesium sulfate. A crude product was obtained after filtration and evaporation, and then purified by silica gel column chromatography to yield an intermediate product H017-1.

The intermediate product H017-1 (15 mmol) and potassium acetate (40 mmol) were mixed with dry 1,4-dioxane (60 mL), Pd(PPh₃)₂Cl₂ (0.4 mmol) and bis(pinacolato)diboron (25 mmol) in a round bottom flask (250 mL). The mixture was stirred at 90° C. for 48 hours under nitrogen atmosphere. The obtained intermediate was cooled to room temperature, added to water, and then filtered through a diatomite pad. The filtrate was extracted with dichloromethane, then washed with water and dried over anhydrous magnesium sulfate. A crude product was obtained after filtration and evaporation, and then purified by silica gel column chromatography to yield an intermediate product H017-2.

The intermediate product H017-2 (10 mmol), 1-chloro-3,5-dipyridylbenzene (12 mmol) and Pd(PPh₃)₄ (0.3 mmol) were added to a mixture of toluene (30 mL)/ethanol (20 mL) and an aqueous solution (10 mL) of potassium carbonate (12 mmol) in a round bottom flask (250 mL). The obtained mixture was refluxed for 12 hours under nitrogen atmosphere, added to water after being cooled to room temperature, and then filtered through a diatomite pad. The filtrate was extracted with dichloromethane, then washed with water and dried over anhydrous magnesium sulfate. A crude product was obtained after filtration and evaporation, and then purified by silica gel column chromatography to yield a final product H017.

Elemental analysis of the Compound H017 (molecular formula C₄₆H₃₉N₃): theoretical values: C, 87.17; H, 6.20; N, 6.63; tested values: C, 87.17; H, 6.19; N, 6.64. Liquid chromatography-mass spectrometry ESI-MS (m/z) (M+): theoretical value: 633.31; tested value: 633.75.

### Example 3

### Synthesis of Compound H041

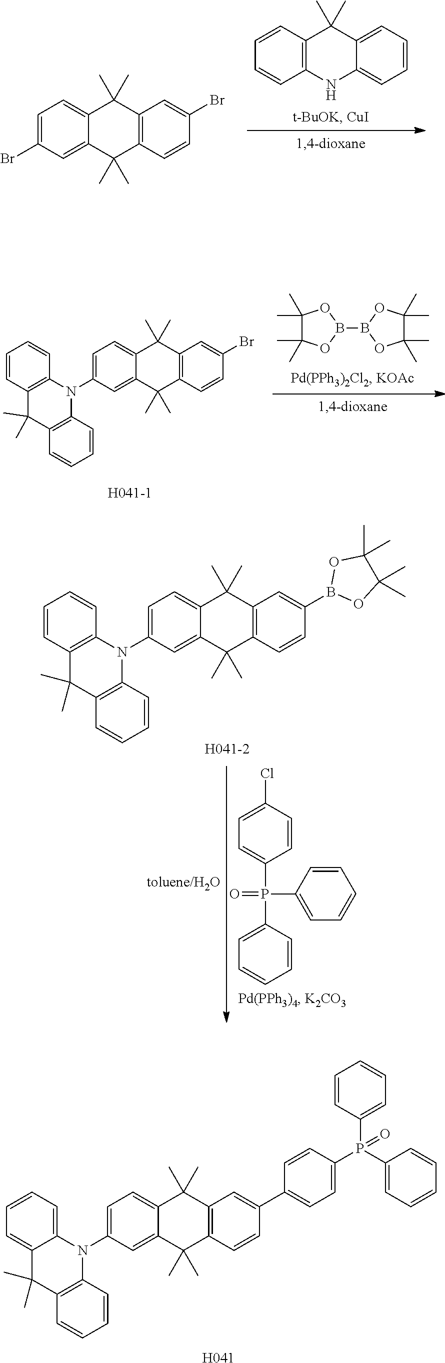

2,6-dibromo-9,9,10,10-tetramethyl-9,10-dihydroanthracene (15 mmol), copper iodide (15 mmol), potassium tert-butoxide (65 mmol), 1,2-diamino cyclohexane (12 mmol) and 9,9-dimethyl-9,10-dihydroacridine (25 mmol) were added to dry 1,4-dioxane (400 mL) in a round bottom flask (250 mL), and the mixture was refluxed under nitrogen atmosphere for 48 hours. The obtained intermediate was cooled to room temperature, added to water, and then filtered through a diatomite pad. The filtrate was extracted with dichloromethane, then washed with water and dried over anhydrous magnesium sulfate. A crude product was obtained after filtration and evaporation, and then purified by silica gel column chromatography to yield an intermediate product H041-1.

The intermediate product H041-1 (15 mmol) and potassium acetate (40 mmol) were mixed with dry 1,4-dioxane (60 mL), Pd(PPh₃)₂Cl₂ (0.4 mmol) and bis(pinacolato)diboron (25 mmol) in a round bottom flask (250 mL). The mixture was stirred at 90° C. for 48 hours under nitrogen atmosphere. The obtained intermediate was cooled to room temperature, added to water, and then filtered through a diatomite pad. The filtrate was extracted with dichloromethane, then washed with water and dried over anhydrous magnesium sulfate. A crude product was obtained after filtration and evaporation, and then purified by silica gel column chromatography to yield an intermediate product H041-2.

The intermediate product H041-2 (10 mmol), 1-chloro-4-(diphenylphosphono)-benzene (12 mmol) and Pd(PPh₃)₄ (0.3 mmol) were added to a mixture of toluene (30 mL)/ethanol (20 mL) and an aqueous solution (10 mL) of potassium carbonate (12 mmol) in a round bottom flask (250 mL). The obtained mixture was refluxed for 12 hours under nitrogen atmosphere, added to water after being cooled to room temperature, and then filtered through a diatomite pad. The filtrate was extracted with dichloromethane, then washed with water and dried over anhydrous magnesium sulfate. A crude product was obtained after filtration and evaporation, and then purified by silica gel column chromatography to yield a final product H041.

Elemental analysis of the Compound H041 (molecular formula C₅₁H₄₆NOP): theoretical values: C, 85.09; H, 6.44; N, 1.95; O, 2.22; P, 4.30; tested values: C, 85.09; H, 6.43; N, 1.96; O, 2.22; P, 4.30. Liquid chromatography-mass spectrometry ESI-MS (m/z) (M+): theoretical value: 719.33; tested value: 719.82.

### Example 4

### Synthesis of Compound H072

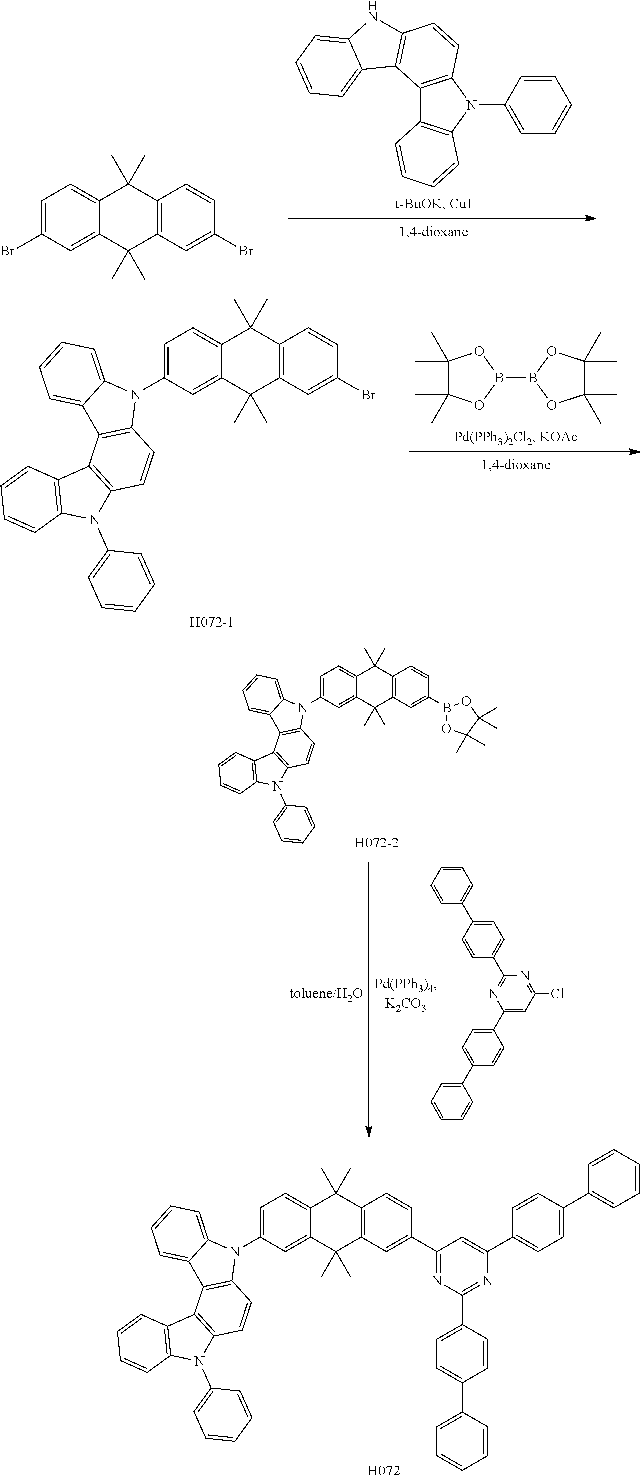

5-phenyl-5,8-dihydro-5,8-azaindole[2,1-c] fluorene (15 mmol), copper iodide (15 mmol), potassium tert-butoxide (65 mmol), 1,2-diamino cyclohexane (12 mmol) and 9,9-dimethyl-9,10-dihydroacridine (25 mmol) were added to dry 1,4-dioxane (400 mL) in a round bottom flask (250 mL), and the mixture was refluxed under nitrogen atmosphere for 48 hours. The obtained intermediate was cooled to room temperature, added to water, and then filtered through a diatomite pad. The filtrate was extracted with dichloromethane, then washed with water and dried over anhydrous magnesium sulfate. A crude product was obtained after filtration and evaporation, and then purified by silica gel column chromatography to yield an intermediate product H072-1.

The intermediate product H072-1 (15 mmol) and potassium acetate (40 mmol) were mixed with dry 1,4-dioxane (60 mL), Pd(PPh₃)₂Cl₂ (0.4 mmol) and bis(pinacolato)diboron (25 mmol) in a round bottom flask (250 mL). The mixture was stirred at 90° C. for 48 hours under nitrogen atmosphere. The obtained intermediate was cooled to room temperature, added to water, and then filtered through a diatomite pad. The filtrate was extracted with dichloromethane, then washed with water and dried over anhydrous magnesium sulfate. A crude product was obtained after filtration and evaporation, and then purified by silica gel column chromatography to yield an intermediate product H072-2.

The intermediate product H072-2 (10 mmol), 4-chrolo-2,6-diphenylpyrimidine (12 mmol) and Pd(PPh₃)₄ (0.3 mmol) were added to a mixture of toluene (30 mL)/ethanol (20 mL) and an aqueous solution (10 mL) of potassium carbonate (12 mmol) in a round bottom flask (250 mL). The obtained mixture was refluxed for 12 hours under nitrogen atmosphere, added to water after being cooled to room temperature, and then filtered through a diatomite pad. The filtrate was extracted with dichloromethane, then washed with water and dried over anhydrous magnesium sulfate. A crude product was obtained after filtration and evaporation, and then purified by silica gel column chromatography to yield a final product H072.

Elemental analysis of the Compound H072 (molecular formula C₇₀H₅₂N₄): theoretical values: C, 88.58; H, 5.52; N, 5.90; tested values: C, 88.58; H, 5.51; N, 5.91. Liquid chromatography-mass spectrometry ESI-MS (m/z) (M+): theoretical value: 948.42; tested value: 948.71.

TABLE 1
Energy level of the exemplary compounds

| Compound | HOMO (eV) | LUMO (eV) | Eg (ev) | E_{T} (ev) |
|---|---|---|---|---|
| H003 | −5.583 | −2.439 | 3.144 | 2.875 |
| H017 | −5.608 | −2.406 | 3.202 | 2.902 |
| H041 | −5.541 | −2.387 | 3.154 | 2.946 |
| H072 | −5.495 | −2.502 | 2.993 | 2.869 |

It can be seen from the above Table 1 that the Compounds H003, H017, H041 and H072, as the host material, show appropriate HOMO and LUMO energy levels and extremely high triplet energy E_{T} (>2.85 ev). Thus, these compounds are suitable to be applied as the host materials of red light (at least E_{T}>2.2 ev), green light (at least E_{T}>2.5 ev), and blue light (at least E_{T}>2.7 ev), and can effectively achieve the energy transfer between the host material and the guest material without the risk of reverse charge transfer.

### Example 5

This example provides an organic light-emitting device. As shown in FIG. 2, the organic light-emitting device includes a glass substrate 1, an ITO anode 2, a first hole transmission layer 3, a second hole transmission layer 4, a light-emitting layer 5, a first electron transmission layer 6, a second electron transmission layer 7, a cathode 8 (magnesium silver electrode with a mass ratio of magnesium to silver of 9:1) and a capping layer (CPL) 9. The ITO anode 2 has a thickness of 15 nm, the first hole transmission layer 3 has a thickness of 10 nm, and the second hole transmission layer 4 has a thickness of 95 nm, the light-emitting layer 5 has a thickness of 30 nm, the first electron transmission layer 6 has a thickness of 35 nm, the second electron transmission layer 7 has a thickness of 5 nm, the magnesium silver electrode 8 has a thickness of 15 nm, and the capping layer (CPL) 9 has a thickness of 100 nm.

The organic light-emitting device of this example was manufactured according to the following steps:

(1) The glass substrate 1 was cut into a size of 50 mm×50 mm×0.7 mm, then subjected to ultrasonic treatment in isopropyl alcohol and deionized water for 30 minutes, respectively, and then exposed to ozone for about 10 minutes for cleaning. The obtained glass substrate with the ITO anode was placed on a vacuum deposition equipment.

(2) A hole transmission layer material HAT-CN was vacuum evaporated onto the ITO anode layer 2 to form the first hole transmission layer 3 having a thickness of 10 nm.

(3) A second hole transmission layer material TAPC was vacuum evaporated onto the first hole transmission layer 3 to form the second hole transmission layer 4 having a thickness of 95 nm.

(4) The light-emitting layer 5 having a thickness of 30 nm was co-deposited on the hole transmission layer 4, where Compound H003 was used as the host material, and Ir(ppy)₃ was used as the doping material with a mass ratio of Compound H003 to Ir(ppy)₃ of 19:1 in the light-emitting layer 5.

(5) A material BPen was vacuum evaporated onto the light-emitting layer 5 to form the first electron transmission layer 6 having a thickness of 30 nm.

(6) A material Alq3 was vacuum evaporated onto the first electron transmission layer 6 to form the second electron transmission layer 7 having a thickness of 5 nm.

(7) The magnesium silver electrode having a thickness of 15 nm, as the cathode 8, was formed on the second electron transmission layer 7 by vacuum evaporating magnesium and silver with a mass ratio of magnesium to silver of 9:1.

(8) A hole type material CBP having a high refraction index was vacuum evaporated onto the cathode 8 to form a cathode covering layer (capping layer or CPL) 9 having a thickness of 100 nm.

The compounds and the structures thereof involved in the present example are shown as follow.

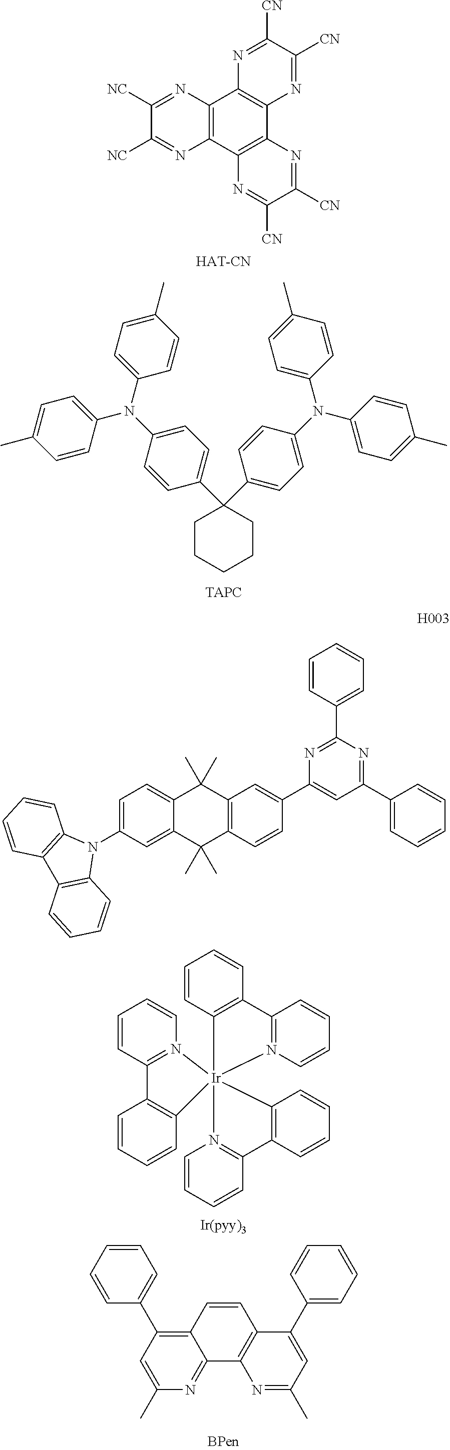

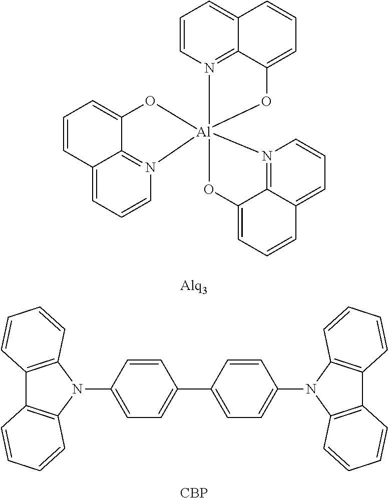

### Example 6

In Example 6, the device was manufactured according to the steps described in Example 5, and the material of each layer was the same except the Compound H017 was used as the host material.

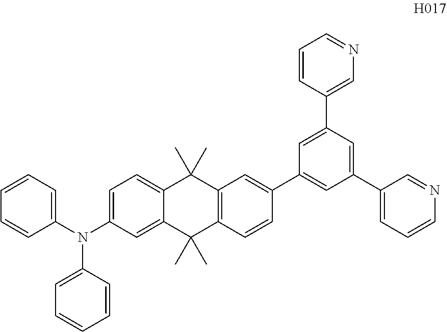 [SMILES: CC1(C)c2ccc(N(c3ccccc3)c3ccccc3)cc2C(C)(C)c2ccc(-c3cc(-c4cccnc4)cc(-c4cccnc4)c3)cc21]

### Example 7

In Example 7, the device was manufactured according to the steps described in Example 5, and the material of each layer was the same except the Compound H041 was used as the host material.

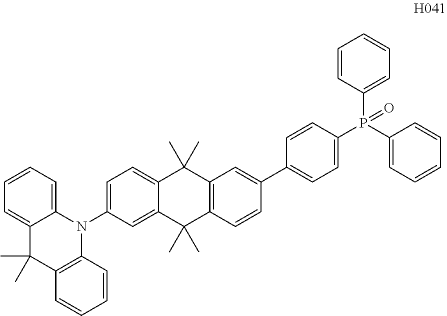 [SMILES: CC1(C)c2ccccc2N(c2ccc3c(c2)C(C)(C)c2ccc(-c4ccc(P(=O)(c5ccccc5)c5ccccc5)cc4)cc2C3(C)C)c2ccccc21]

### Example 8

In Example 8, the device was manufactured according to the steps described in Example 5, and the material of each layer was the same except the Compound H072 was used as the host material.

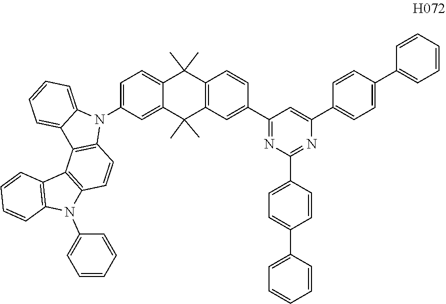

### Comparative Example 1

In Comparative Example 1, the device was manufactured according to the steps described in Example 5, and the material of each layer was the same except the host material was CzTRZ.

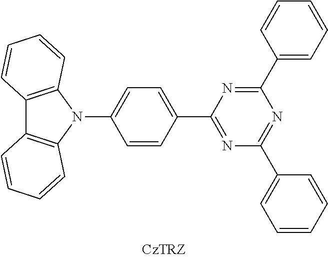 [SMILES: c1ccc(-c2nc(-c3ccccc3)nc(-c3ccc(-n4c5ccccc5c5ccccc54)cc3)n2)cc1]

TABLE 2
Performance characterization of devices

| | | driving voltage | | CE |
|---|---|---|---|---|
| No. | host material | (V) | EQE/% | (cd/A) |
| Example 5 | H003 | 3.80 | 28.2% | 118.9 |
| Example 6 | H017 | 3.82 | 31.3% | 125.7 |
| Example 7 | H041 | 3.79 | 29.7% | 120.1 |
| Example 8 | H072 | 3.86 | 30.6% | 123.8 |
| Comparative | CzTRZ | 4.10 | 24.2% | 103.2 |
| Example 1 | | | | |

It can be seen from Table 2 that the driving voltages of the light-emitting devices adopting the compounds of the present disclosure are about 8.5% lower than the driving voltage of the device in the comparative example 1, so that power consumption of the devices can be effectively reduced. The luminous efficiency of the light-emitting devices using the compounds of the present disclosure as the host material is improved by about 10%-25%, thereby effectively improving the brightness and service life of the devices.

In another example, the present disclosure provides a display panel including the above-mentioned organic light-emitting device.

In still another example, the present disclosure provides a display apparatus including the above-mentioned display panel.

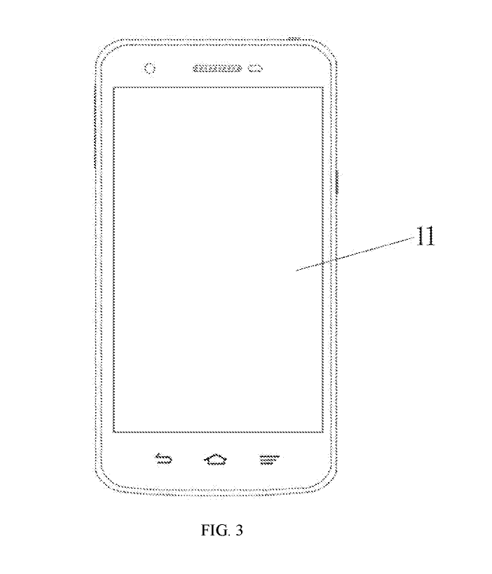

In the present disclosure, the organic light-emitting device may be an OLED used in an organic light-emitting display apparatus. The organic light-emitting display apparatus can be display screen of various smart devices, such a mobile phone display screen, a computer display screen, a liquid crystal television display screen, a smart watch display screen, a display panel of smart car, a display screen of Virtual Reality (VR) or Augmented Reality (AR), etc. FIG. 3 is a schematic diagram of a display apparatus according to an embodiment of the present disclosure, in which 11 denotes a mobile phone display screen.

What is claimed is:

1. A compound having a chemical structure represented by Formula (I): 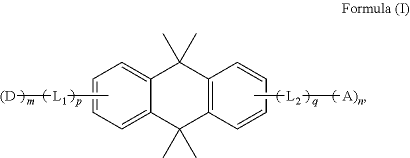
wherein D represents an electron donor, A represents an electron acceptor, m is a number of the electron donor D, n is a number of the electron acceptor A, and m and n are each independently 1, 2, or 3,
p is a number of the group L₁, q is a number of the group L₂, and p and q are each independently 0, 1, or 2,
L₁ and L₂ are each independently selected from the group consisting of a single bond, a substituted or unsubstituted C1-C20 alkylene, a substituted or unsubstituted C3-C20 cycloalkylene, a substituted or unsubstituted C3-C20 heterocycloalkylene, a substituted or unsubstituted C6-C40 arylene, a substituted or unsubstituted C4-C40 heteroarylene, a substituted or unsubstituted C10-C60 fused arylene, and a substituted or unsubstituted C10-C60 fused heteroarylene,
the electron donor D is selected from the group consisting of a substituted or unsubstituted C1-C20 alkyl, a substituted or unsubstituted C3-C20 cycloalkyl, a substituted or unsubstituted C1-C20 alkoxy, a substituted or unsubstituted C3-C20 heterocyclic group, a substituted or unsubstituted C6-C40 aryl, a substituted or unsubstituted C4-C40 heteroaryl, a substituted or unsubstituted C10-C60 fused arylene, a substituted or unsubstituted C10-C60 fused heteroarylene, a substituted or unsubstituted C12-C40 carbazolyl and a derivative group thereof, a substituted or unsubstituted C12-C40 diphenylamino and a derivative group thereof, and a substituted or unsubstituted C13-C40 acridinyl and a derivative group thereof, and
the electron acceptor A is selected from the group consisting of nitrogen-containing heterocyclic substituents, cyano-containing substituents, triaryl-boron-derived substituents, and phosphorus oxygen double bond phosphorus oxygen double bond-containing substituents.

2. The compound according to claim 1, wherein the electron donor D is selected from the following groups: 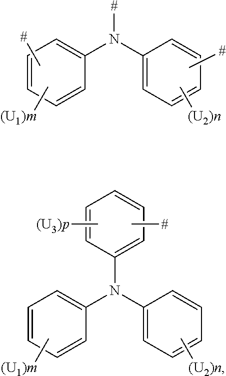
wherein m, n and p are each independently 0, 1, 2 or 3,
U₁, U₂ and U₃ are each independently selected from the group consisting of hydrogen, a substituted or unsubstituted C1-C30 alkyl, a substituted or unsubstituted silicylene, a substituted or unsubstituted C3-C20 cycloalkyl, a substituted or unsubstituted C1-C30 alkoxy, a substituted or unsubstituted C6-C30 aryl, and a substituted or unsubstituted C10-C30 fused aryl, and
# represents a bonding position.

3. The compound according to claim 2, wherein the electron donor D is selected from the following groups: 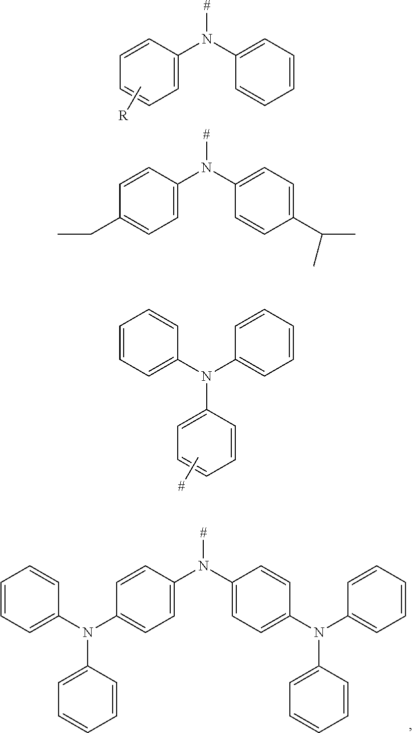
wherein R is selected from the group consisting of hydrogen, a substituted or unsubstituted C1-C20 alkyl, a substituted or unsubstituted silicylene, a substituted or unsubstituted C3-C20 cycloalkyl, a substituted or unsubstituted C1-C20 alkoxy, a substituted or unsubstituted C3-C20 heterocyclic group, a substituted or unsubstituted C6-C40 aryl, a substituted or unsubstituted C10-C30 fused aryl, and a substituted or unsubstituted C4-C40 heteroaryl.

4. The compound according to claim 1, wherein the electron donor D is selected from the following groups: 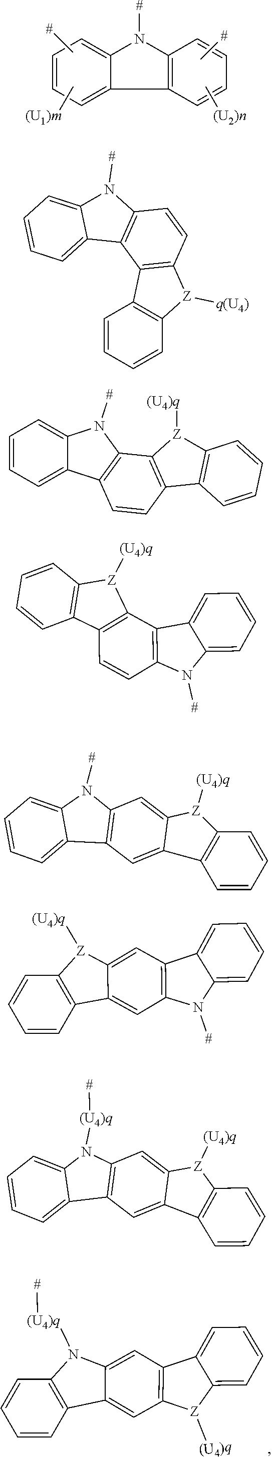
wherein Z is carbon, nitrogen, oxygen, sulfur, or silicon,
q is 0, 1, 2, or 3,
U₁, U₂ and U₄ are each independently selected from the group consisting of hydrogen, a substituted or unsubstituted C1-C30 alkyl, a substituted or unsubstituted silicylene, a substituted or unsubstituted C3-C20 cycloalkyl, a substituted or unsubstituted C1-C30 alkoxy, a substituted or unsubstituted C6-C30 aryl, and a substituted or unsubstituted C10-C30 fused aryl,
when Z is oxygen or sulfur, q is 0, and
# represents a bonding position.

5. The compound according to claim 4, wherein the electron donor D is selected from the following groups:

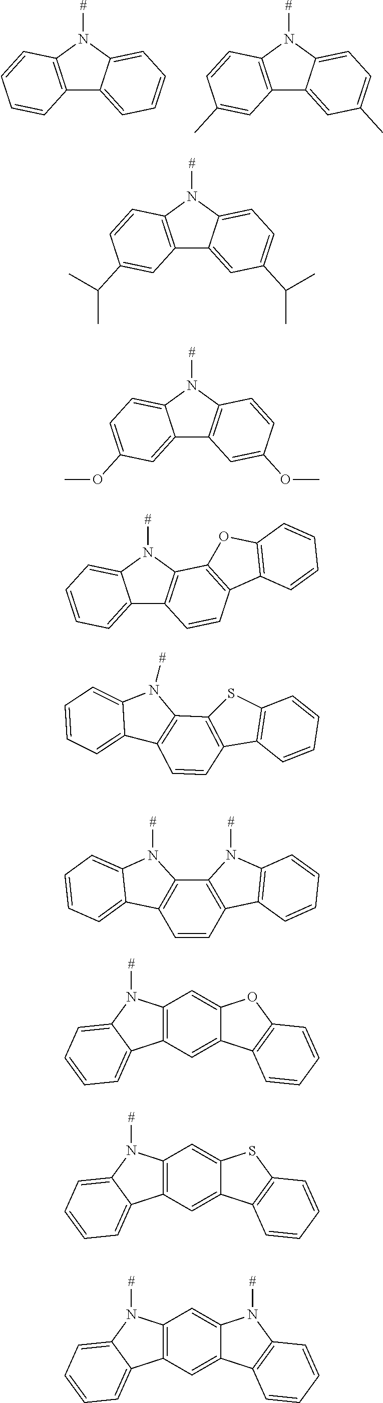

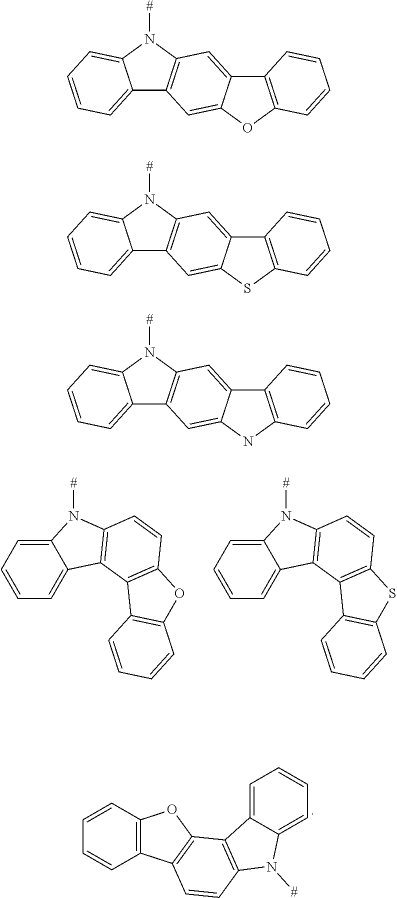

6. The compound according to claim 1, wherein the electron donor D is selected from the following groups: 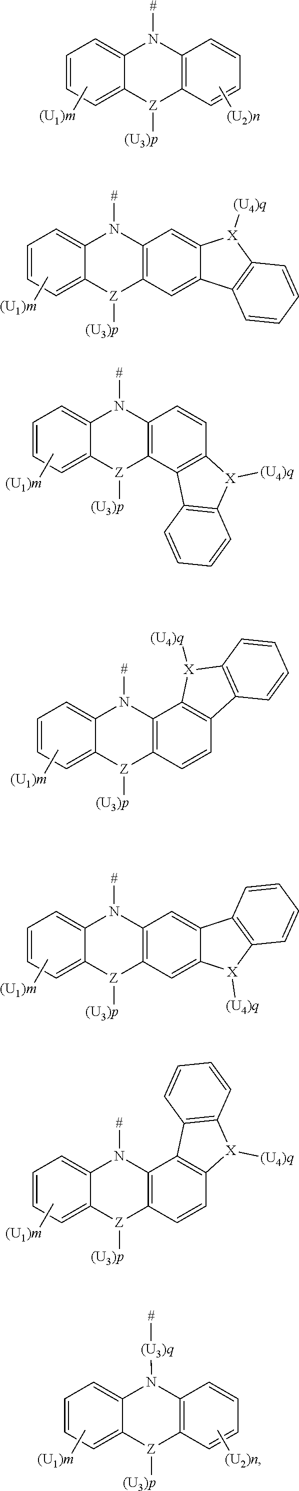
wherein Z is carbon, nitrogen, oxygen, sulfur or silicon,
X is carbon, nitrogen, oxygen, or sulfur,
m, n, p and p are each independently 0, 1, 2, or 3,
U₁, U₂, U₃ and U₄ are each independently selected from the group consisting of hydrogen, a substituted or unsubstituted C1-C30 alkyl, a substituted or unsubstituted silicylene, a substituted or unsubstituted C3-C20 cycloalkyl, a substituted or unsubstituted C1-C30 alkoxy, a substituted or unsubstituted C6-C30 aryl, and a substituted or unsubstituted C10-C30 fused aryl,
when Z is oxygen or sulfur, p is 0,
when X is oxygen or sulfur, q is 0, and
# represents a bonding position.

7. The compound according to claim 6, wherein the electron donor D is selected from the following groups: 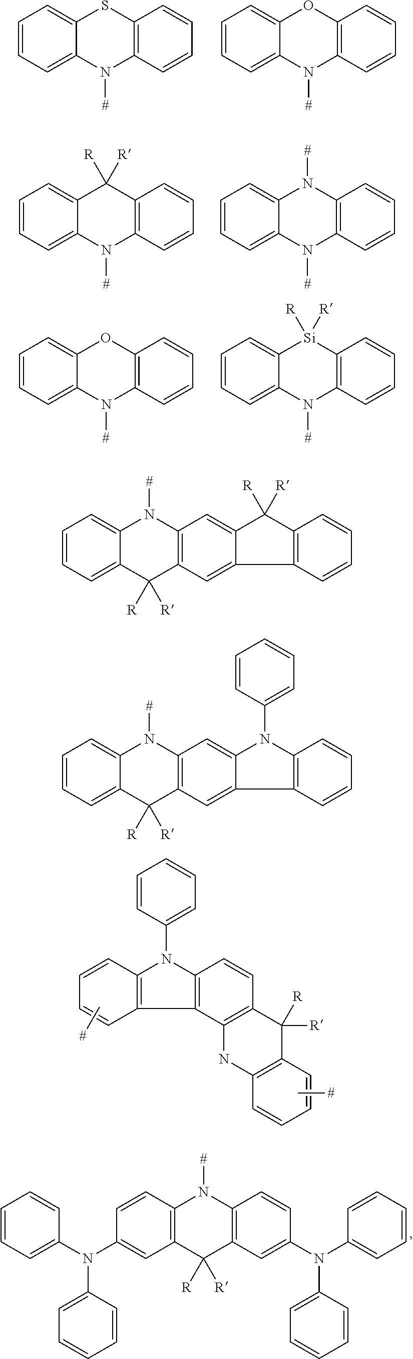
wherein R and R′ are each independently selected from the group consisting of hydrogen, a substituted or unsubstituted C1-C20 alkyl, a substituted or unsubstituted C3-C20 cycloalkyl, a substituted or unsubstituted C1-C20 alkoxy, a substituted or unsubstituted C3-C20 heterocyclic group, a substituted or unsubstituted C6-C40 aryl, and a substituted or unsubstituted C4-C40 heteroaryl.

8. The compound according to claim 1, wherein the electron acceptor A is selected from the following groups: 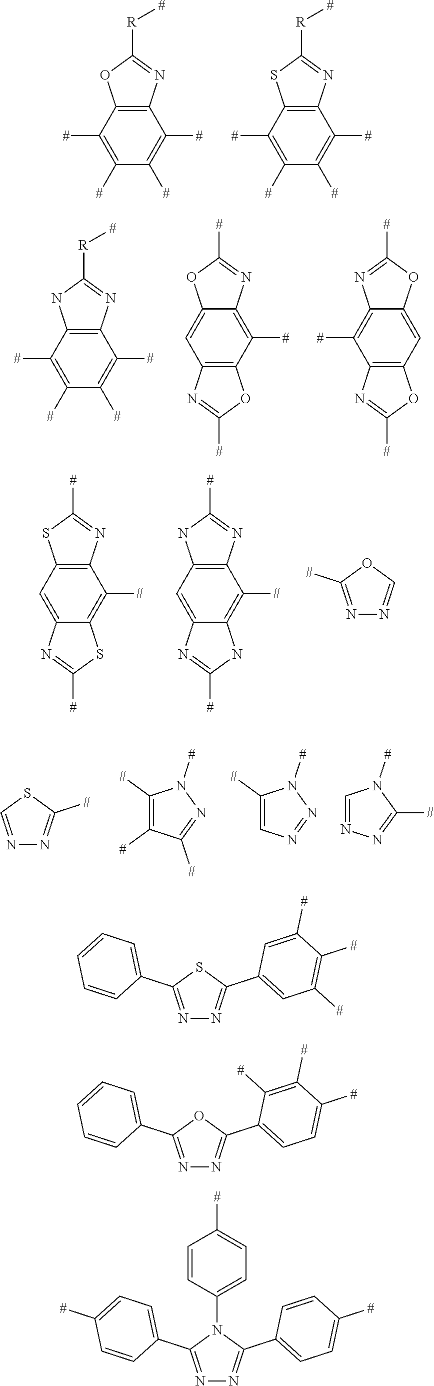 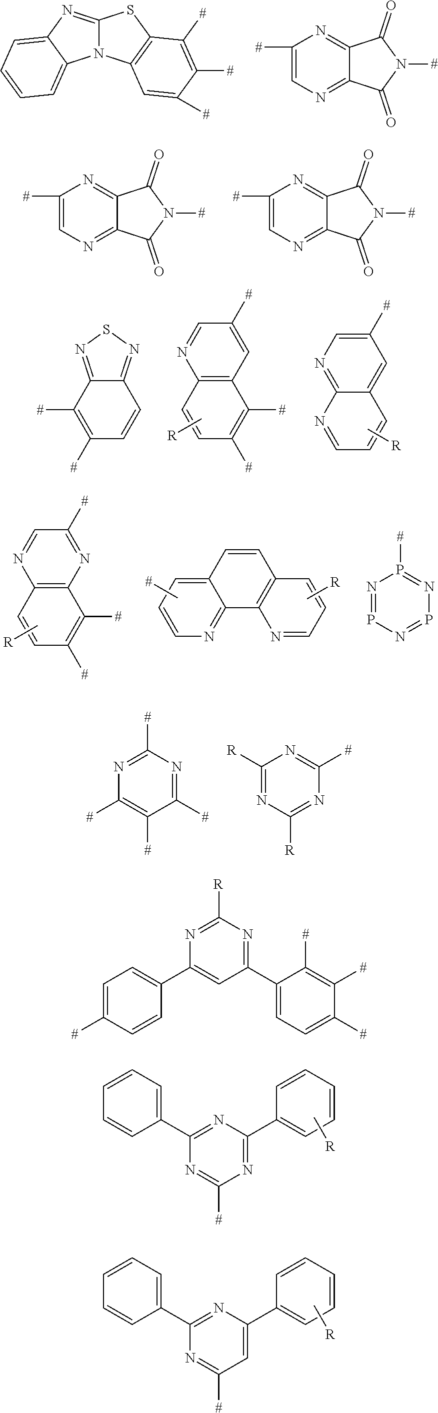 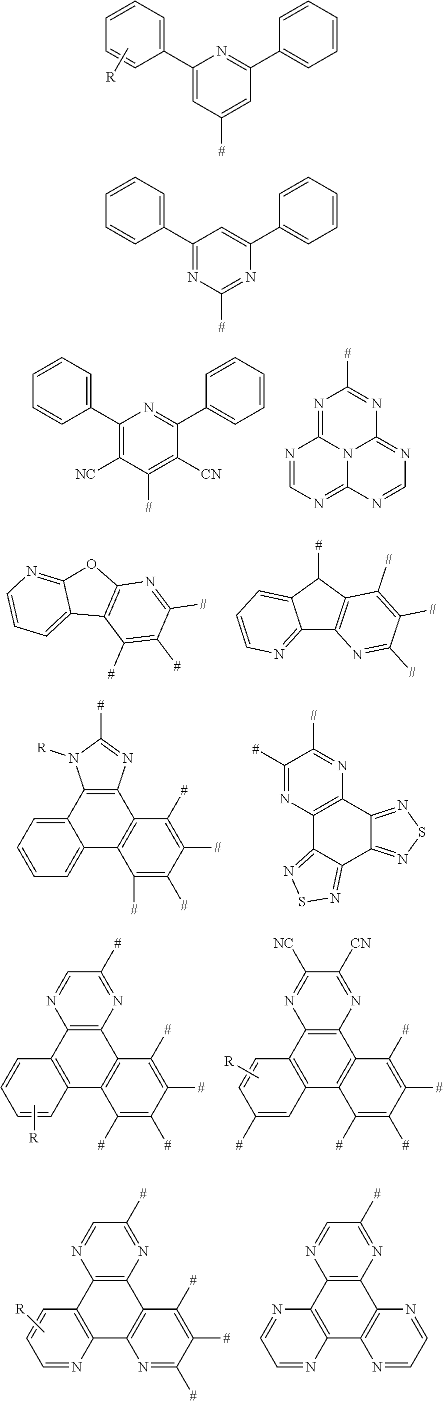 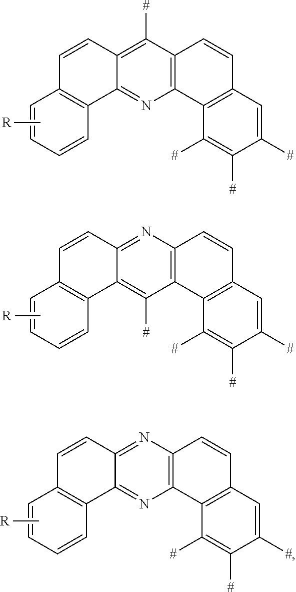
wherein R is hydrogen, a C1-C20 alkyl, a C1-C20 alkoxy, a C4-C8 cycloalkyl, a C6-C40 aryl, or a C4-C40 heteroaryl, and
# represents a bonding position.

9. The compound according to claim 1, wherein the electron acceptor A is selected from the following groups: 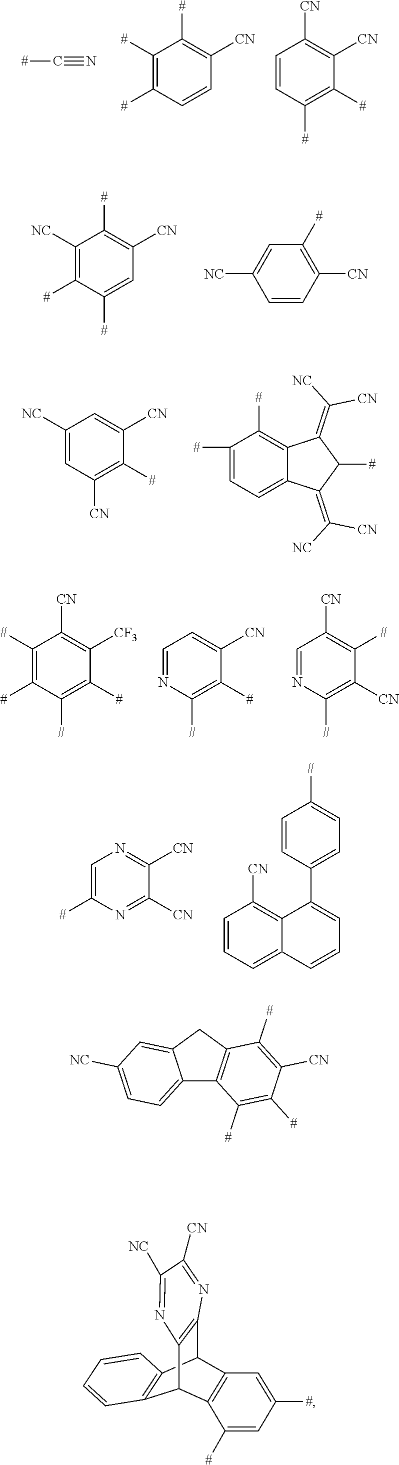
wherein # represents a bonding position.

10. The compound according to claim 1, wherein the electron acceptor A is selected from the following groups: 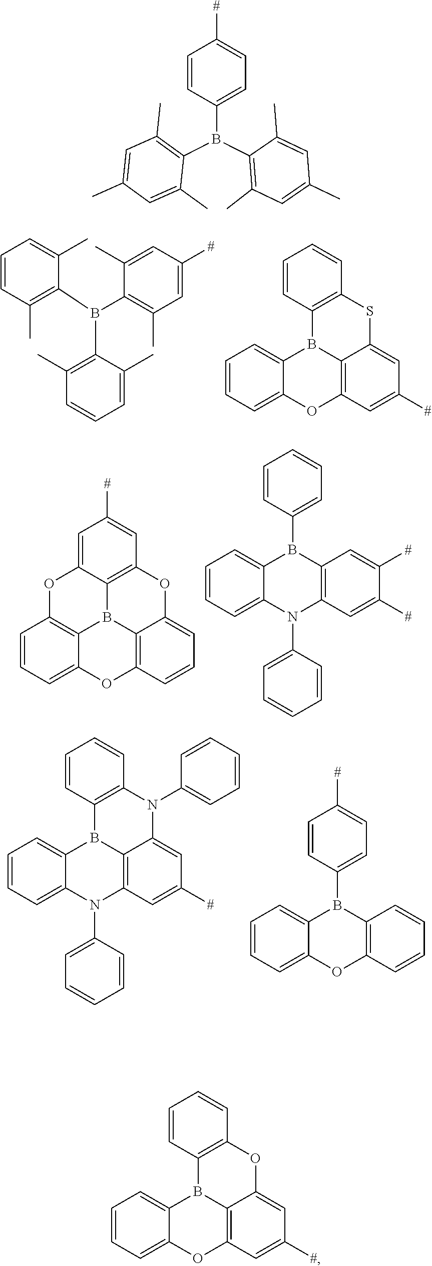
wherein # represents a bonding position.

11. The compound according to claim 1, wherein the electron acceptor A is selected from the following groups: 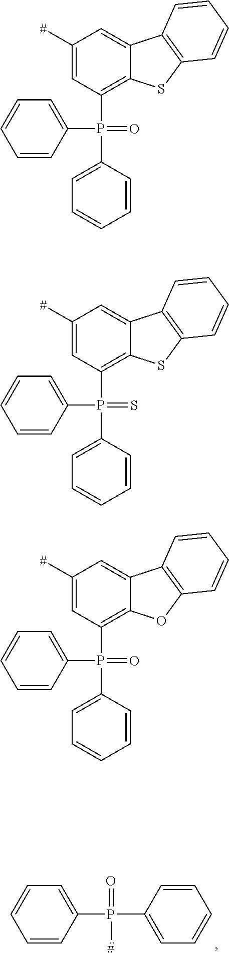
wherein # represents a bonding position.

12. The compound according to claim 1, wherein the compound is selected from the following compounds:

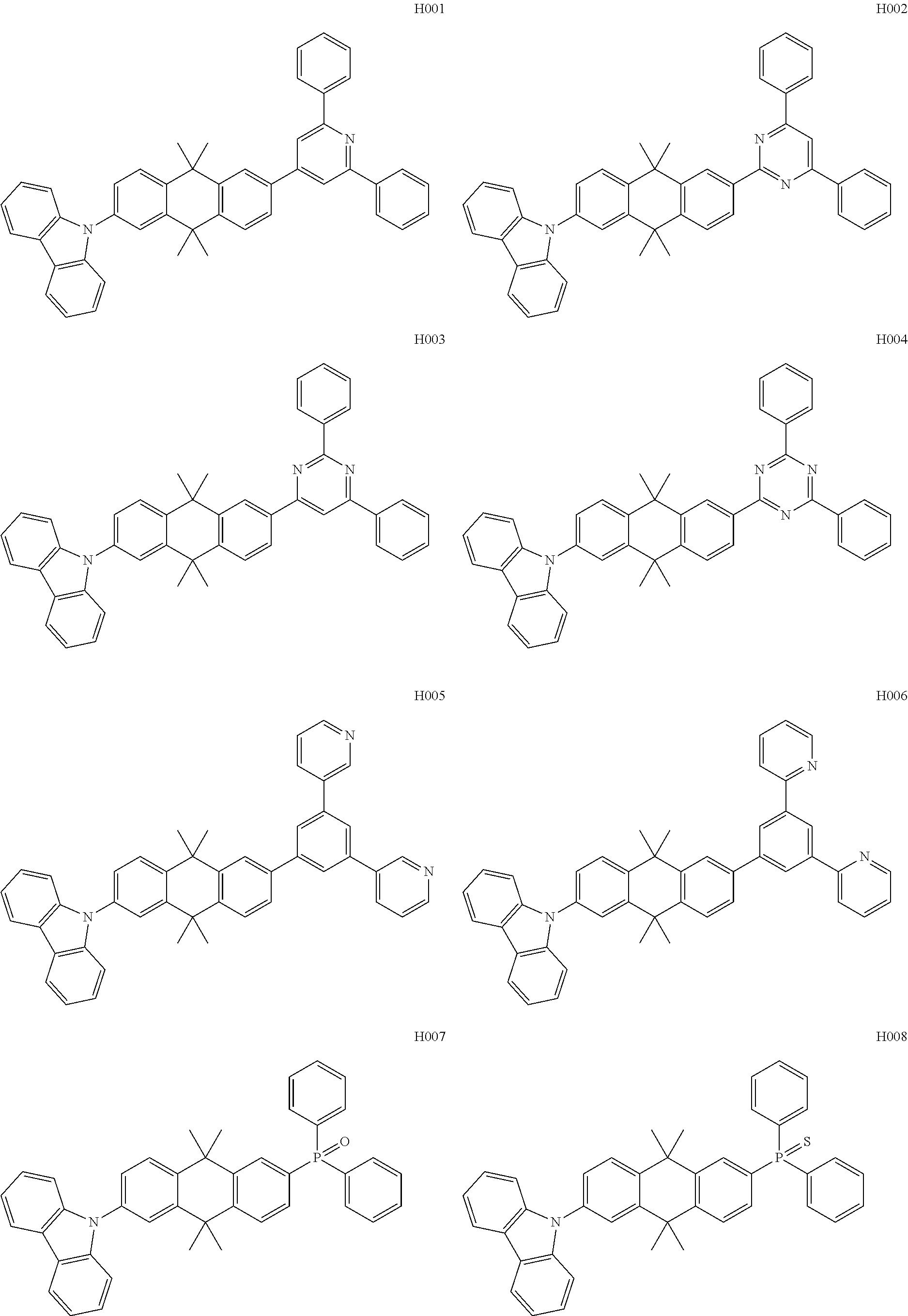

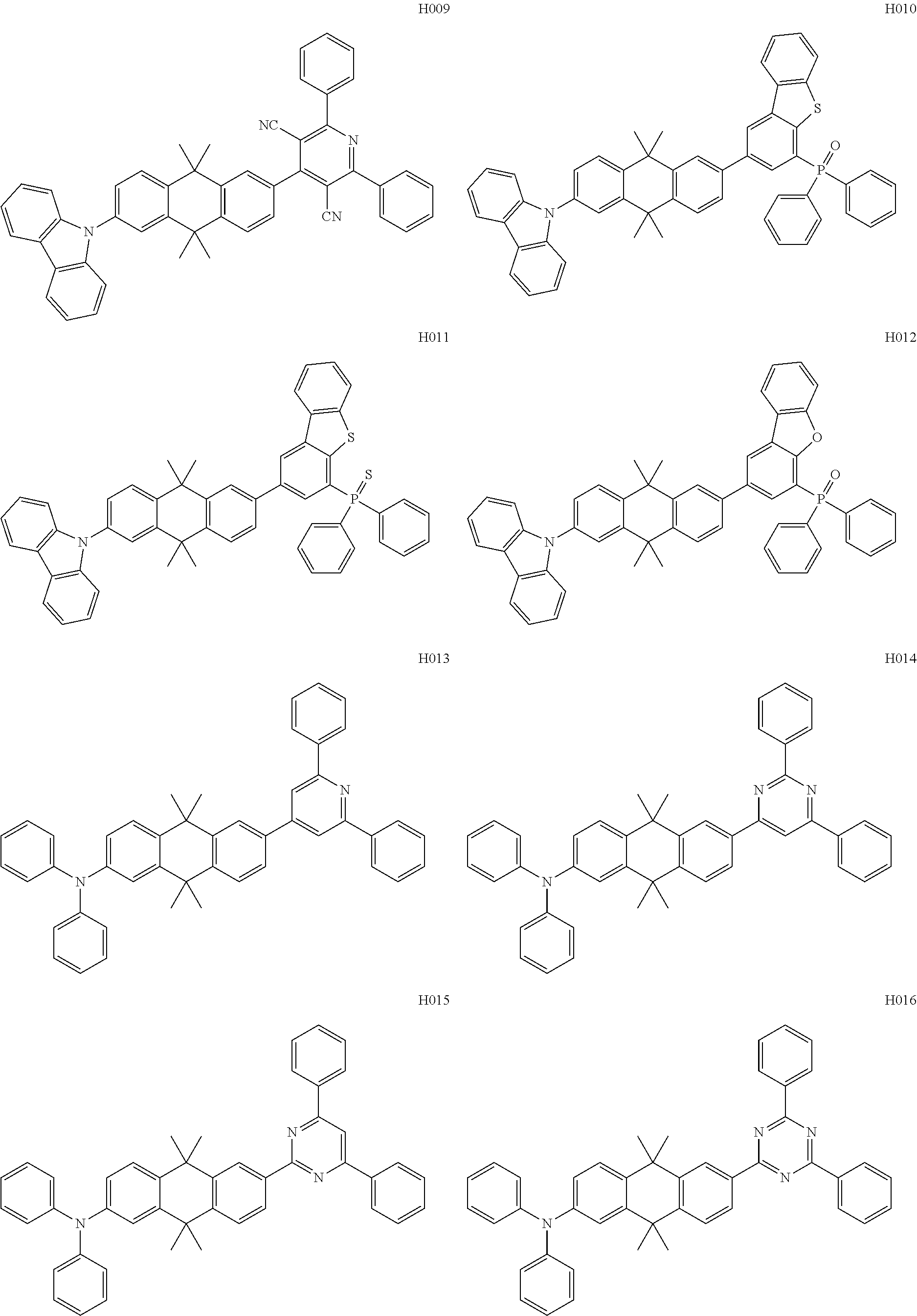

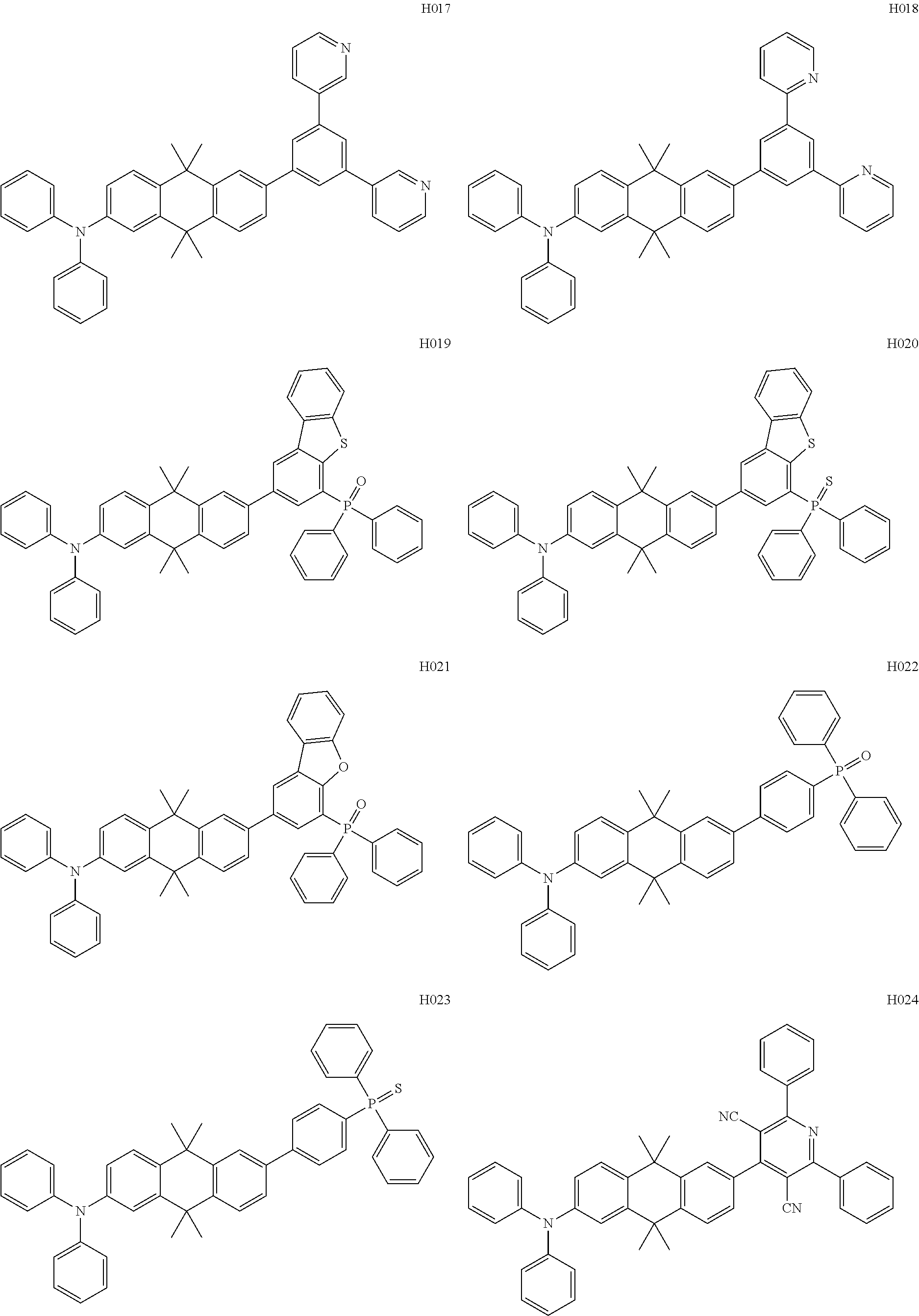

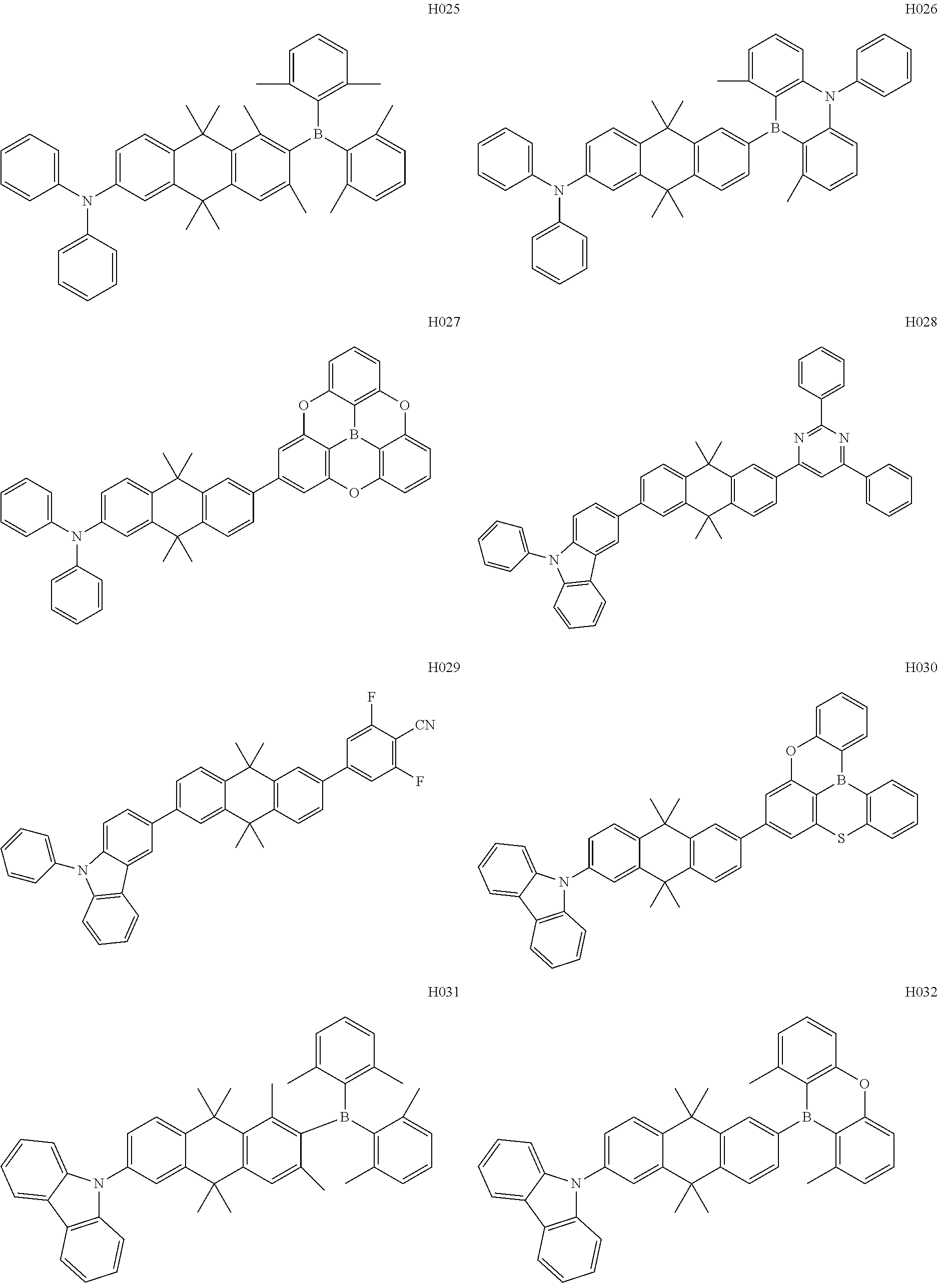

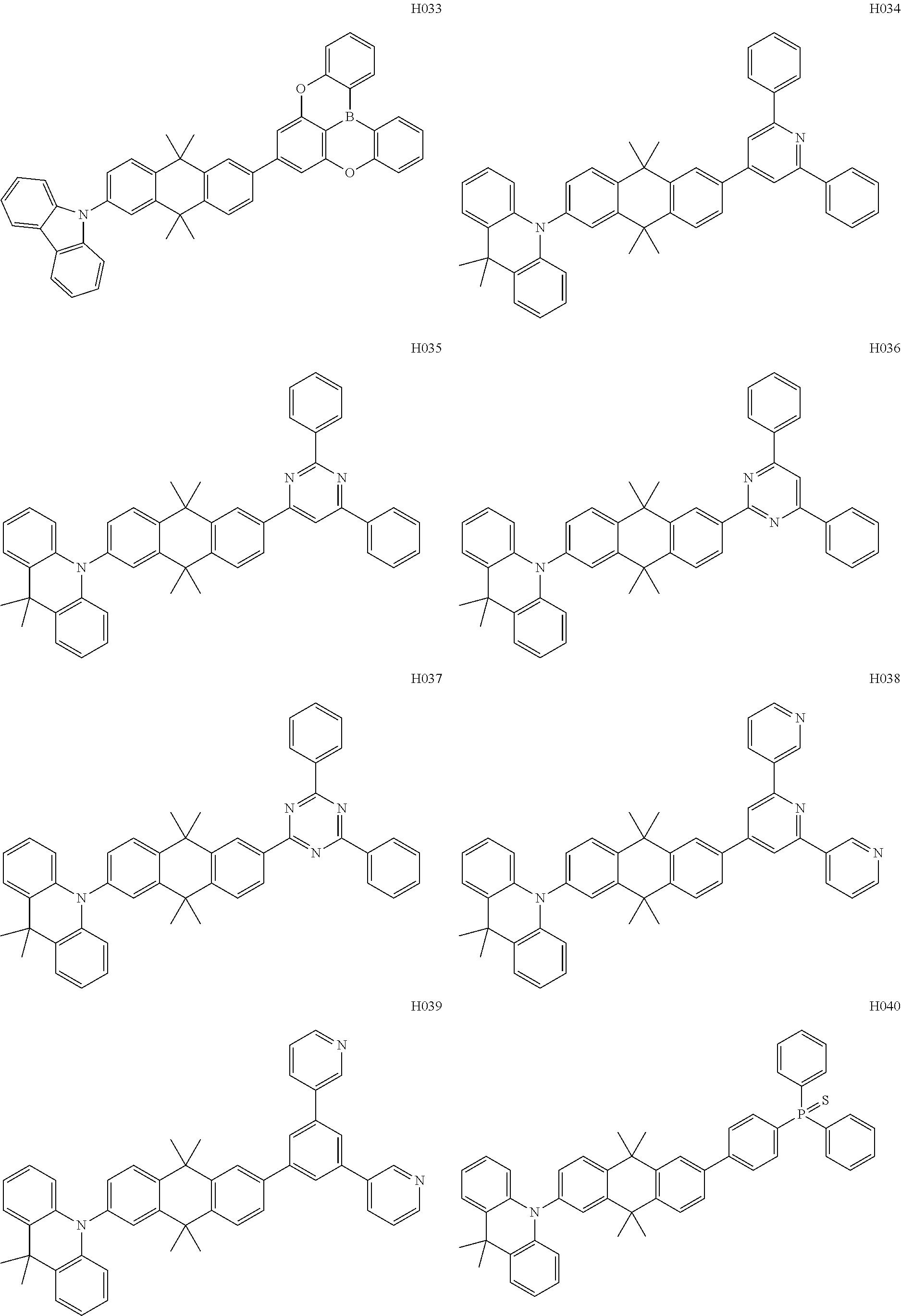

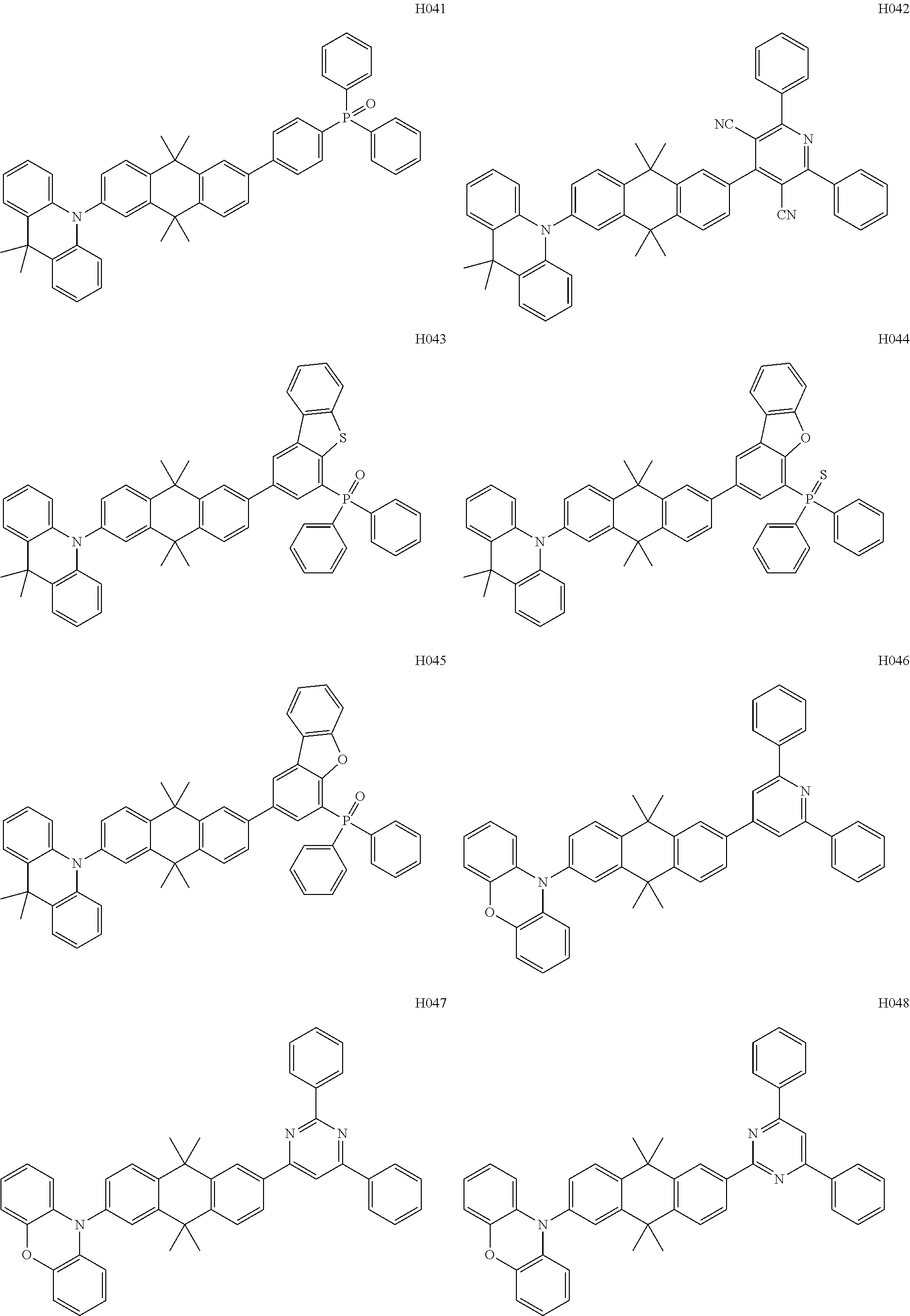

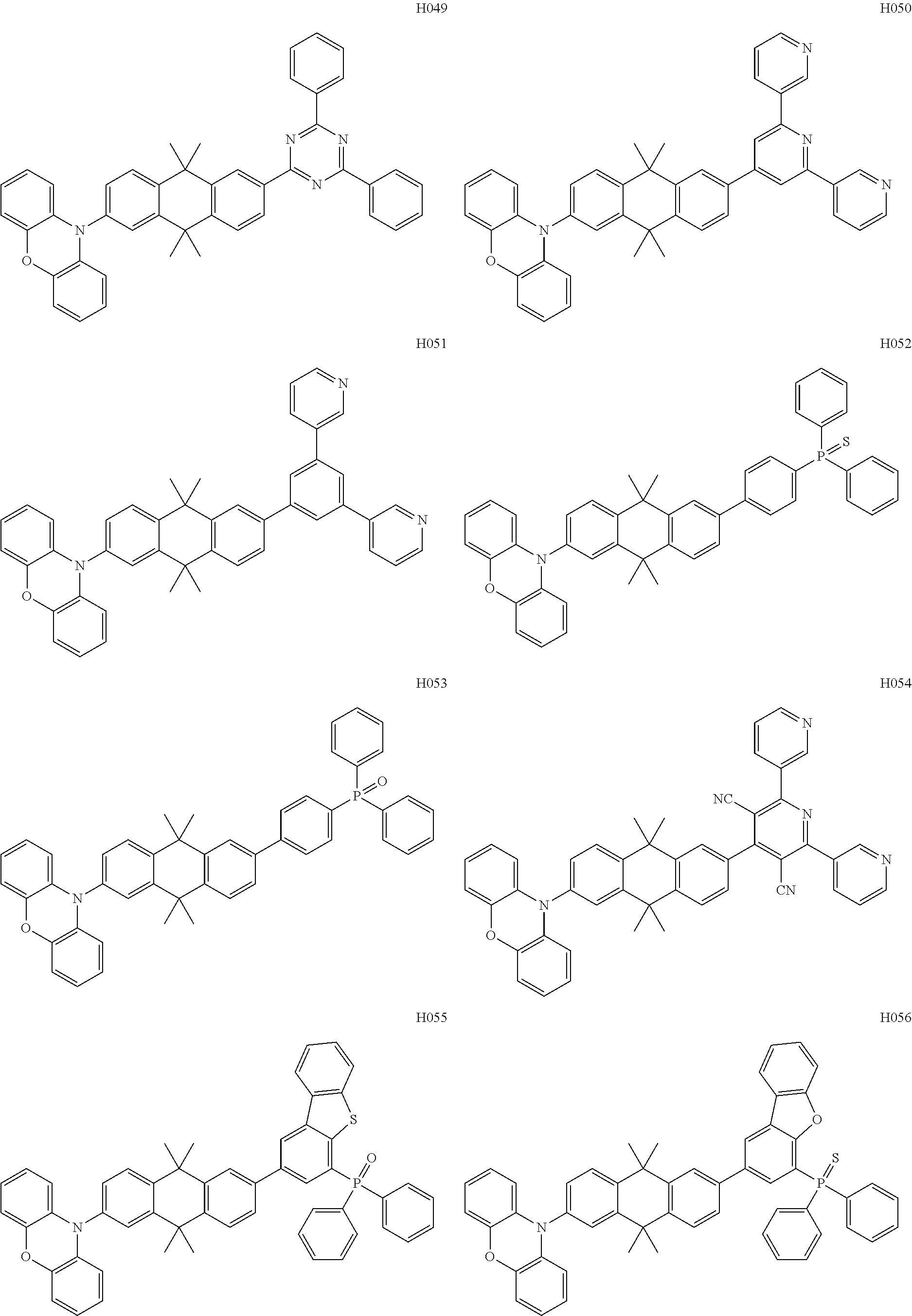

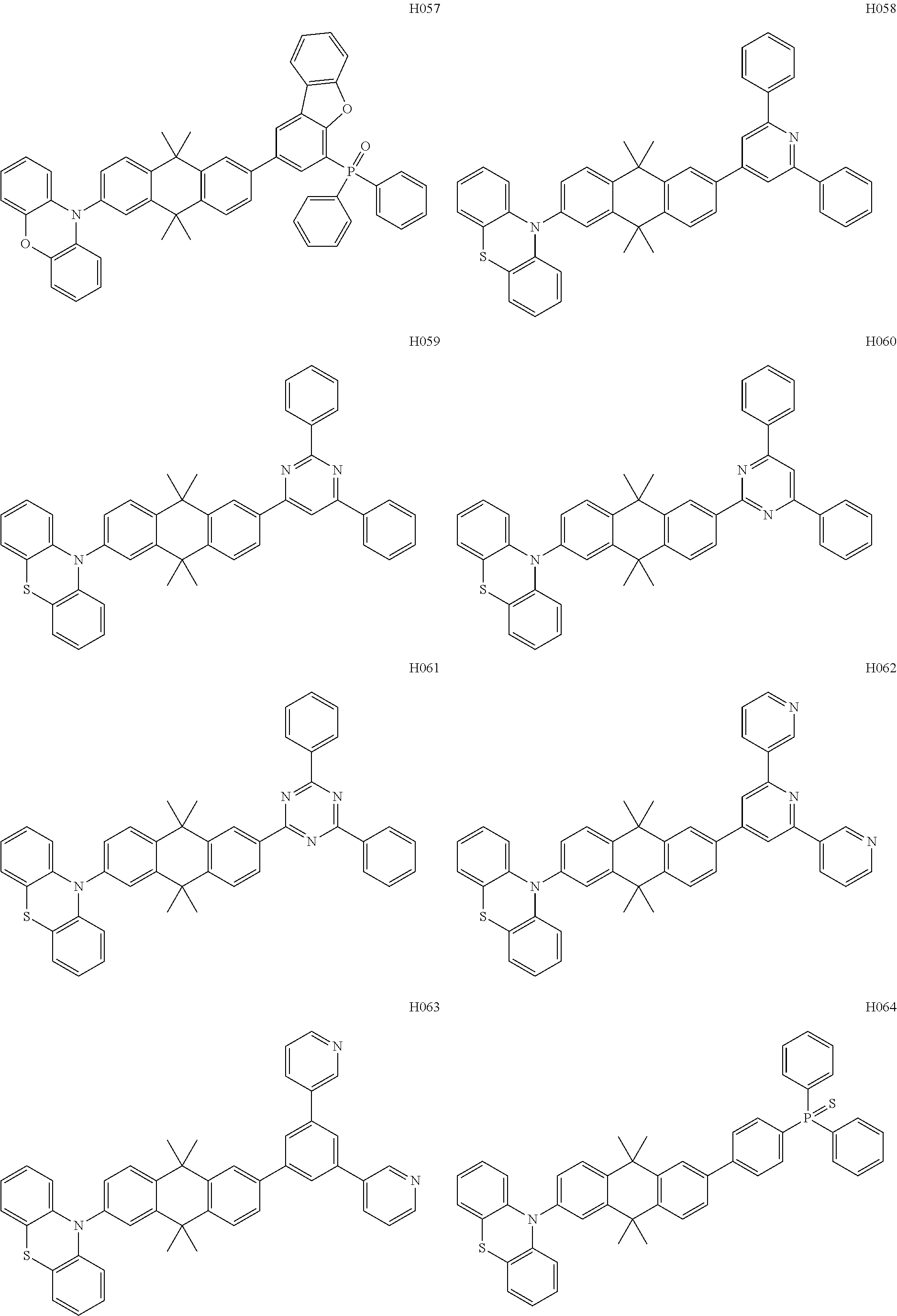

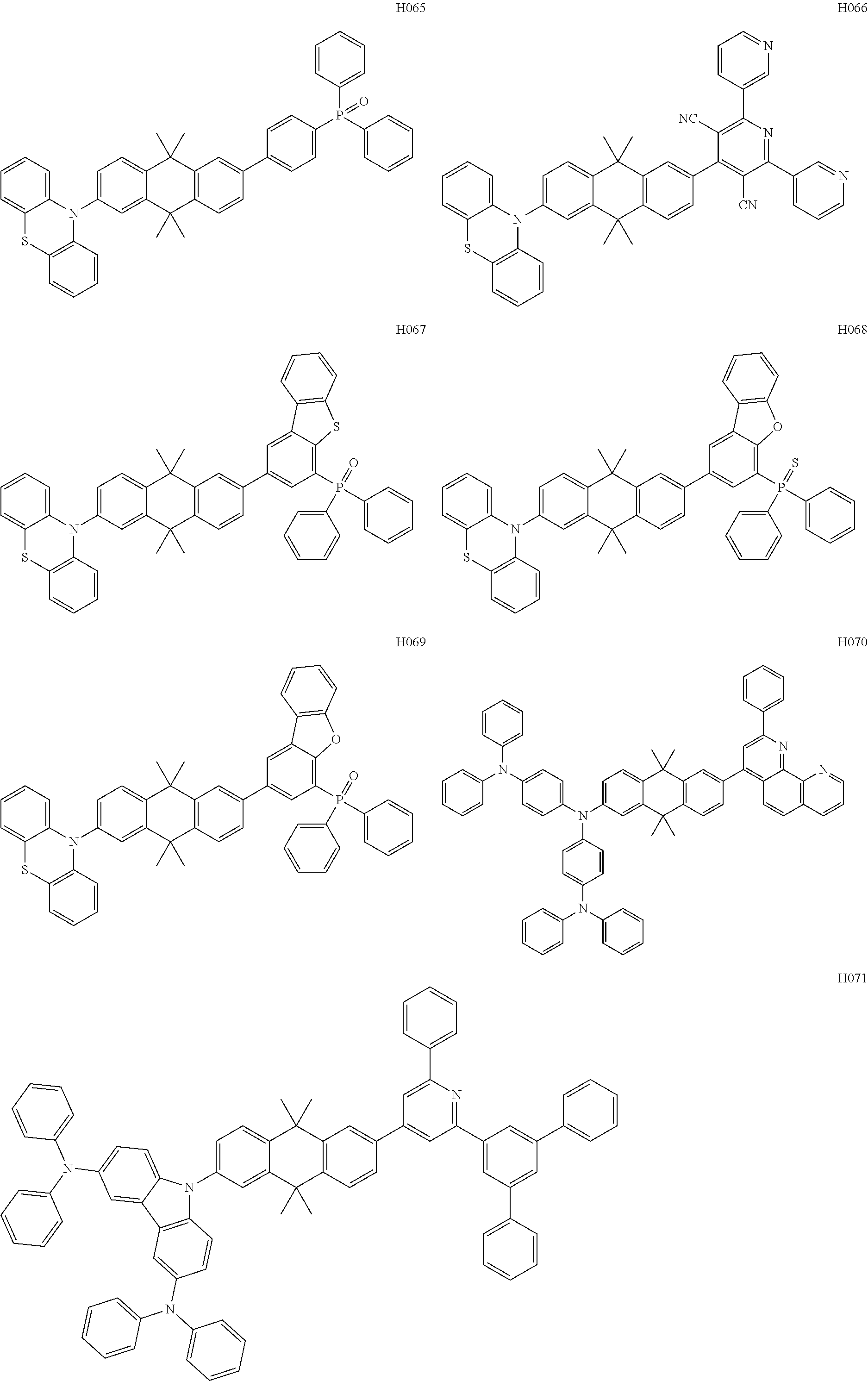

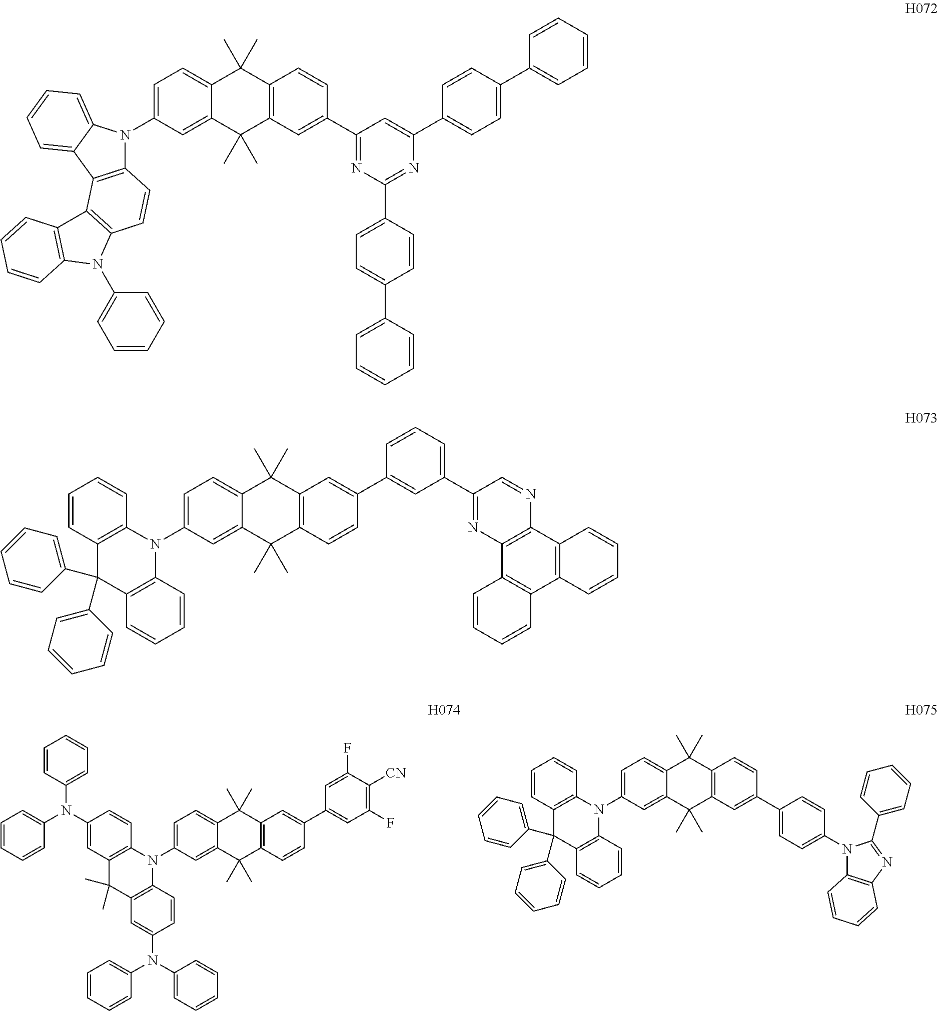 [SMILES: CC1(C)c2ccc(-c3cc(-c4ccc(-c5ccccc5)cc4)nc(-c4ccc(-c5ccccc5)cc4)n3)cc2C(C)(C)c2cc(-n3c4ccccc4c4c5c6ccccc6n(-c6ccccc6)c5ccc43)ccc21 ; CC1(C)c2ccc(N3c4ccccc4C(c4ccccc4)(c4ccccc4)c4ccccc43)cc2C(C)(C)c2ccc(-c3cccc(-c4cnc5c6ccccc6c6ccccc6c5n4)c3)cc21 ; CC1(C)c2cc(N(c3ccccc3)c3ccccc3)ccc2N(c2ccc3c(c2)C(C)(C)c2ccc(-c4cc(F)c(C#N)c(F)c4)cc2C3(C)C)c2ccc(N(c3ccccc3)c3ccccc3)cc21 ; CC1(C)c2ccc(-c3ccc(-n4c(-c5ccccc5)nc5ccccc54)cc3)cc2C(C)(C)c2cc(N3c4ccccc4C(c4ccccc4)(c4ccccc4)c4ccccc43)ccc21]

13. A display panel, comprising an organic light-emitting device, wherein the organic light-emitting device comprises an anode, a cathode disposed oppositely to the anode, and a light-emitting layer disposed between the anode and the cathode, wherein
the light-emitting layer comprises a host material and a guest material, and
the host material is one or more compounds according to claim 1.

14. The display panel according to claim 13, wherein a singlet energy level S1 of the host material is higher than a singlet energy level S1 of the guest material, and an energy difference between the singlet energy level S1 of the host material and the singlet energy level S1 of the guest material is less than 0.8 eV, and
wherein a triplet energy level T1 of the host material is higher than a triplet energy level T1 of the guest material, and an energy difference between the triplet energy level T1 of the host material and the triplet energy level T1 of the guest material is less than 0.4 eV.

15. The display panel according to claim 13, wherein when the host material of the light-emitting layer is a red-light-emitting material, a triplet energy level T1 of the red-light-emitting material has a lowest value as 2.2 eV;
when the host material of the light-emitting layer is a green-light-emitting material, a triplet energy level T1 of the green-light-emitting material has a lowest value as 2.5 eV; and
when the host material of the light-emitting layer is a blue-light-emitting material, a triplet energy level T1 of the blue-light-emitting material has a lowest value as 2.7 eV.

16. The display panel according to claim 13, wherein the organic light-emitting device comprises:
a capping layer, and the capping layer is disposed on a side of the cathode facing away from the anode;
at least one of a hole injection layer, a hole transmission layer, an electron blocking layer, a hole blocking layer, an electron transmission layer, and an electron injection layer.

17. The display panel according to claim 13, wherein
the capping layer, and at least one of the electron transmission layer, the hole transmission layer and the light-emitting layer, are made of the compound according to claim 1.

18. A display apparatus, comprising the display panel according to claim 13.
